(19) United States
(12) Patent Application Publication
(10) Pub. No.: US 2014/0121594 A1
(43) Pub. Date: May 1, 2014

(54) Implantable Tastemaker for Automatic Taste Modification of Selected Foods

(71) Applicant: Robert A. Connor, Forest Lake, MN (US)

(72) Inventor: Robert A. Connor, Forest Lake, MN (US)

(21) Appl. No.: 13/661,502

(22) Filed: Oct. 26, 2012

(51) Int. Cl. A61M 37/00

(52) U.S. Cl. 604/77

(57) ABSTRACT

This invention is a device and method for automatic and selective modification of the taste and/or smell of one or more selected nutrient or food types as food is being digested within a person's mouth. This invention can comprise: a taste and/or smell modifying substance; a reservoir for this substance that is implanted within the person's body; and a flow control mechanism that automatically and selectively increases flowable communication between this substance and the person's oral cavity or nasal cavity when a selected nutrient or food is being digested within the person's mouth. This invention can help a person to moderate their consumption of unhealthy food and to limit their overall caloric intake as part of a comprehensive system for improved nutrition, energy balance, and weight management.

### CROSS-REFERENCE TO RELATED APPLICATIONS

This patent application claims the priority benefit of U.S. Provisional Patent Application No. 61/714,896 entitled “Implantable Tastemaker for Automatic Taste Modification of Selected Foods” filed on Oct. 17, 2012 by Robert A. Connor of Medibotics, LLC.

### FEDERALLY SPONSORED RESEARCH

Not Applicable

### SEQUENCE LISTING OR PROGRAM

Not Applicable

### BACKGROUND

### Field of Invention

This invention relates to proper nutrition, energy balance, and weight management.

### Introduction to Nutrition and Caloric Intake Management

The United States population has some of the highest prevalence rates of obese and overweight people in the world. Further, these rates have increased dramatically during recent decades. In the late 1990's, around one in five Americans was obese. Today, that figure has increased to around one in three. It is estimated that around one in five American children is now obese. The prevalence of Americans who are generally overweight is estimated to be as high as two out of three.

This increase in the prevalence of Americans who are overweight or obese has become one of the most common causes of health problems in the United States. Potential adverse health effects from obesity include: cancer (especially endometrial, breast, prostate, and colon cancers); cardiovascular disease (including heart attack and arterial sclerosis); diabetes (type 2); digestive diseases; gallbladder disease; hypertension; kidney failure; obstructive sleep apnea; orthopedic complications; osteoarthritis; respiratory problems; stroke; metabolic syndrome (including hypertension, abnormal lipid levels, and high blood sugar); impairment of quality of life in general including stigma and discrimination; and even death.

There are estimated to be over a quarter-million obesity-related deaths each year in the United States. The tangible costs to American society of obesity have been estimated at over $100 billion dollars per year. This does not include the intangible costs of human pain and suffering. Despite the considerable effort that has been focused on developing new approaches for preventing and treating obesity, the problem is growing. There remains a serious unmet need for new ways to help people to moderate their consumption of unhealthy food, better manage their energy balance, and lose weight in a healthy and sustainable manner.

Obesity is a complex disorder with multiple interacting causal factors including genetic factors, environmental factors, and behavioral factors. A person's behavioral factors include the person's caloric intake (the types and quantities of food which the person consumes) and caloric expenditure (the calories that the person burns in regular activities and exercise). Energy balance is the net difference between caloric intake and caloric expenditure. Other factors being equal, energy balance surplus (caloric intake greater than caloric expenditure) causes weight gain and energy balance deficit (caloric intake less than caloric expenditure) causes weight loss.

Since many factors contribute to obesity, good approaches to weight management are comprehensive in nature. Proper nutrition and management of caloric intake are key parts of a comprehensive approach to weight management. Consumption of “junk food” that is high in simple sugars and saturated fats has increased dramatically during the past couple decades, particularly in the United States. This has contributed significantly to the obesity epidemic. For many people, relying on willpower and dieting is not sufficient to moderate their consumption of unhealthy “junk food.” The results are dire consequences for their health and well-being.

The invention that is disclosed herein directly addresses this problem by helping a person to selectively reduce their consumption of unhealthy nutrient and food types. The invention that is disclosed herein is an innovative technology that can be a key part of a comprehensive system that helps a person to reduce their consumption of unhealthy food, to better manage their energy balance, and to lose weight in a healthy and sustainable manner. In the following sections, we categorize and review the prior art, provide a summary of this invention and its advantages over the prior art, and then provide some detailed examples of how this invention can be embodied to help a person to improve their nutrition and to manage their weight.

### Categorization and Review of the Prior Art

It can be challenging to classify prior art into discrete categories. This is the certainly the case in the field of nutrition improvement, energy balance, and weight management. There are numerous examples of potentially-relevant prior art. However, classification of the prior art into categories, even if imperfect, is an invaluable tool for reviewing the prior art, identifying its limitations, and setting the stage for discussion of the advantages of the invention that is disclosed in subsequent sections. Towards this end, I now identify 21 general categories of prior art and list examples of prior art which appear to be best classified into each category. This categorization and discussion of the prior art helps to identify limitations of the prior art which are corrected by the invention disclosed herein in subsequent sections.

The 21 categories of prior art that I will now discuss are as follows: (1) food additive, (2) lipstick or toothpaste, (3) tablet, lozenge, or gum, (4) dissolvable film, (5) oral or nasal cavity adhesive patch, (6) dental appliance, (7) oral or nasal spray, (8) limiting mouth capacity or function, (9) non-consumption-related mouth implant, (10) implantable drug pump, (11) implantable glucose sensor, (12) sound sensor to detect consumption, (13) digestive tract sensor, (14) restricting food volume or absorption, (15) applying electricity to digestive tract organs, (16) stimulating salivation, (17) diversion of enzyme secretion, (18) general feedback and support systems for energy balance, (19) methods to identify compounds or substances, (20) specific compounds or substances, and (21) miscellaneous.

The first nine (1-9) categories relate generally to devices and methods concerning the person's oral cavity and nasal cavity (mouth and nose), including substances added to food or applied to the mouth and/or nose and devices attached to, or implanted within, the person's oral cavity or nasal cavity. The next nine (10-18) categories relate generally to devices and methods concerning the rest of the person's digestive tract and nerves leading to and from the digestive tract, including devices and methods attached to, or implanted within the person's stomach and esophagus.

The next two (19-20) categories relate generally to specific substances and compounds that are intended to affect eating habits, including substances and compounds added to food, consumed with food, or administered as a pharmaceutical. The last category (21) is a miscellaneous category for potentially-relevant devices and methods that do not fit well into any of the above categories.

### 1. Food Additive

This category of prior art includes food additives to change a person's food consumption. A food additive can: change the flavor or smell of food to dampen or enhance a person's appetite; dampen or otherwise modify a person's sense of taste or smell; or have a systemic effect that changes a person's appetite. In an example, a food additive can be an ingredient that is included in food during its preparation. In an example, a food additive can be something that is sprinkled on, or otherwise added to, food immediately before a person consumes it.

In order for this approach to be effective, the additive must really change a person's food consumption. There are some food additives for which scientific evidence of consumption modification is lacking. Also, in order for this approach to be effective, either the person must be restricted to eat only food that has been prepared with this additive or the person must be consistent in their voluntary application of this additive whenever they eat (the selected type of) food. However, if a person does not have enough willpower and discipline to avoid eating unhealthy food in the first place, then it is not clear that this person would have enough willpower and discipline to always sprinkle an appetite-suppressing additive on their food each time that they eat.

Examples of prior art that appear to be best classified into this category include: U.S. Pat. No. 5,603,971 (Porzio et al., Feb. 18, 1997, “Encapsulation Compositions”), U.S. Pat. No. 6,112,749 (Hall et al., Sep. 5, 2000, “Flavor Dot Odorizer and Method”), U.S. Pat. No. 6,902,751 (Schleifenbaum et al., Jun. 7, 2005, “Encapsulated Flavorings”), U.S. Pat. No. 7,727,546 (Moneymaker et al., Jun. 1, 2010, “Nutrient System for Individualized Responsive Dosing Regimens”), U.S. Pat. No. 7,820,208 (Hirsch, Oct. 26, 2010, “Method of Assaying Satiety Enhancing Tastants”), U.S. Pat. No. 8,143,062 (Hirsch, Mar. 27, 2012, “Method and Composition for Enhancing Weight Loss”), and U.S. Pat. No. 8,143,215 (Hirsch, Mar. 27, 2012, “Method of Promoting Weight Loss”); and U.S. Patent Applications 20040231299 (Yakushigawa et al., Nov. 25, 2004, “Flavoring System and Method”), 20080075813 (Smith et al., Mar. 27, 2008, “Seasoning and Method for Enhancing and Potentiating Food Flavor Utilizing Microencapsulation . . . ”), 20090123380 (Hirsch, May 14, 2009, “Method of Assaying Satiety Enhancing Tastants”), 20090123524 (Hirsch, May 14, 2009, “Packaged Satiety Enhancing Composition”), 20090123579 (Hirsch, May 14, 2009, “Method of Promoting Weight Loss”), 20090214445 (Boghani et al., Aug. 27, 2009, “Delivery Systems for Managing Release of Functional Ingredients in an Edible Composition”), and 20120058217 (Patty, Mar. 8, 2012, “Taste Deterrent and Diet Method”).

### 2. Lipstick or Toothpaste

This category of prior art includes lipstick or toothpaste that releases a consumption-modifying substance. In order to be effective, the lipstick or toothpaste must release a genuinely consumption-modifying substance in sufficient amounts over a long-enough duration to affect food consumption. If it only releases the substance for a short time or tapers off rapidly, then the lipstick or toothpaste must be applied frequently which relies heavily on the person's voluntary compliance. If it releases the substance for a long time, then the prior art does not disclose how this approach would enable selective consumption modification of unhealthy food. It would affect consumption of healthy foods as well as unhealthy foods. In order for this approach to be effective: the substance in the lipstick or toothpaste must really reduce food consumption when used; the substance must be released from the lipstick or toothpaste in sufficient quantity, and over a sufficient duration, to be effective; and the person must have consistent voluntary compliance in using the lipstick or toothpaste.

Examples of prior art that appear to be best classified into this category include: U.S. Pat. No. 6,485,710 (Zuckerman, Nov. 26, 2002, “Appetite Suppressant Toothpaste”) and U.S. Pat. No. 7,247,323 (George et al., Jul. 24, 2007, “Delivery System for Appetite Suppressant”); and U.S. Patent Applications 20030095936 (Light, May 22, 2003, “Lip Gloss Composition”), 20070042058 (George et al., Feb. 22, 2007, “Delivery System for Appetite Suppressant”), and 20100135945 (Murdock et al., Jun. 3, 2010, “Gymnema-Containing Lip Balm Compositions and Associated Method”).

### 3. Tablet, Lozenge, or Gum

This category of prior art includes tablets, lozenges, and chewing gum that are inserted into the mouth and slowly release a consumption-modifying substance. Since inserting and ingesting the tablet, lozenge, or chewing gum can interfere with the process of food consumption, the person must have sufficient willpower and discipline to insert the tablet, lozenge, or chewing gum well in advance of eating. Further, if the substance in the mouth is diluted by food consumption, then the person would have to insert a tablet, lozenge, or chewing gum multiple times during the same meal. In order for this approach to work, the person must exercise consistent voluntary compliance in inserting the tablet, lozenge, or chewing gum into their mouth before eating (selected types of) food. However, if a person does not have enough willpower and discipline to avoid eating unhealthy food in the first place, then it is not clear that this person would have enough willpower and discipline to consistently pop a tablet, lozenge, or chewing gum into their mouth before each snack or meal.

Examples of prior art that appear to be best classified into this category include: U.S. Pat. No. 3,856,942 (Murphy, Dec. 24, 1974, “Appetite Control Composition”), U.S. Pat. No. 3,911,099 (DeFoney et al., Oct. 7, 1975, “Long-Acting Articles for Oral Delivery and Process”), U.S. Pat. No. 4,039,653 (DeFoney et al., Aug. 2, 1977, “Long-Acting Articles for Oral Delivery and Process”), U.S. Pat. No. 4,822,597 (Faust et al., Apr. 18, 1989, “Anesthetic-Containing Chewing Gum Compositions”), U.S. Pat. No. 5,942,244 (Friedman et al., Aug. 24, 1999, “Local Oral Herbal Slow Release Tablets”), U.S. Pat. No. 6,183,775 (Ventouras, Feb. 6, 2001, “Buccal Delivery System”), U.S. Pat. No. 6,280,761 (Santus, Aug. 28, 2001, “Nicotine Lozenge”), U.S. Pat. No. 6,893,654 (Pinney et al., May 17, 2005, “Two-Stage Transmucosal Medicine Delivery System for Symptom Relief”), U.S. Pat. No. 6,949,264 (McGrew et al., Sep. 27, 2005, “Nutraceuticals or Nutritional Supplements and Method of Making”), U.S. Pat. No. 7,851,000 (Boghani et al., Dec. 14, 2010, “Taste Potentiator Compositions and Edible Confectionery and Chewing Gum Products Containing Same”), and U.S. Pat. No. 8,236,348 (Gin et al., Aug. 7, 2012, “Long-Lasting, Flavored Dosage Forms for Sustained Release of Beneficial Agents within the Mouth”).

Examples of prior art that appear to be best classified into this category also include: U.S. Patent Applications 20040151771 (Gin et al., Aug. 5, 2004, “Long-Lasting, Flavored Dosage Forms for Sustained Release of Beneficial Agents Within the Mouth”), 20040247669 (Gin et al., Dec. 9, 2004, “Long-Lasting Flavored Dosage Forms for Sustained Release of Beneficial Agents within the Mouth”), 20050112149 (Belote et al., May 26, 2005, “Single-Dose Taste Inhibitor Units”), 20070048369 (Foreman et al., Mar. 1, 2007, “Mucosal Delivery Tablet”), 20090081291 (Gin et al., Mar. 26, 2009, “Sustained Release Dosage Forms for Delivery of Agents to an Oral Cavity of a User”), and 20120195954 (Maynard, Aug. 2, 2012, “Method of Reducing Appetite”).

### 4. Dissolvable Film

This category of prior art includes dissolvable films that are inserted into the mouth and slowly release a consumption-modifying substance. Since inserting and ingesting the film can interfere with the process of food consumption, the person must have sufficient willpower and discipline to insert the film in advance of eating. Further, if the substance in the mouth is diluted by food consumption, then the person would have to insert a dissolvable film multiple times during the same meal. In order for this approach to work, the person must exercise consistent voluntary compliance in inserting the film into their mouth before eating (selected types of) food. However, if a person does not have enough willpower and discipline to avoid eating unhealthy food in the first place, then it is not clear that this person would have enough willpower and discipline to consistently insert a dissolvable film into their mouth before each snack or meal.

Examples of prior art that appear to be best classified into this category include U.S. Pat. No. 6,419,903 (Xu et al., Jul. 16, 2002, “Breath Freshening Film”) and U.S. Pat. No. 7,972,618 (Fuisz et al., Jul. 5, 2011, “Edible Water-Soluble Film Containing a Foam Reducing Flavoring Agent”); and U.S. Patent Applications 20040131661 (Auffret et al., Jul. 8, 2004, “Process for Making Orally Consumable Dosage Forms”) and 20120109051 (Harrell, May 3, 2012, “Devices, Methods, and Kits for Taste Modification and Controlling Food Intake”).

### 5. Oral or Nasal Cavity Adhesive Patch

This category of prior art includes adhesive patches (or strips) that are applied to the interior surface of a person's oral or nasal cavity. The patch (or strip) then gradually releases a consumption-modifying substance into the body tissue or into the cavity itself In an example, this substance can be absorbed into tissue, such as through mucosal delivery, to cause a systemic (pharmacological) appetite-suppressant effect. In an example, this substance can be released into the person's oral cavity or nasal cavity to cause a localized anesthetic effect.

If the effect of the patch lasts for a short time, then the patch must be replaced frequently, which requires high voluntary compliance by the person. If the effect lasts for a long time, then the prior art does not disclose how this approach would enable selective consumption modification (allowing healthy food but discouraging unhealthy food). Also, the prior art does not disclose how such a patch (or strip) could allow moderate consumption of certain foods but limit excess consumption of those foods.

Examples of prior art that appear to be best classified into this category include: U.S. Pat. No. 3,972,995 (Tsuk et al., Aug. 3, 1976, “Dosage Form”), U.S. Pat. No. 4,059,686 (Tanaka et al., Nov. 22, 1977, “Pharmaceutical Preparation for Oral Cavity Administration”), U.S. Pat. No. 4,292,299 (Suzuki et al., Sep. 29, 1981, “Slow-Releasing Medical Preparation to be Administered by Adhering to a Wet Mucous Surface”), U.S. Pat. No. 4,615,697 (Robinson, Oct. 7, 1986, “Bioadhesive Compositions and Methods of Treatment Therewith”), U.S. Pat. No. 4,764,378 (Keith et al., Aug. 16, 1988, “Buccal Drug Dosage Form”), U.S. Pat. No. 6,387,408 (Illum et al., May 14, 2002, “Adhesive Drug Delivery Composition”), U.S. Pat. No. 6,488,953 (Halliday et al., Dec. 3, 2002, “Oral Transmucosal Delivery”), and U.S. Pat. No. 8,173,113 (Scholz et al., May 8, 2012, “Bioadhesive Composition and Patch”); and U.S. Patent Applications 20040109886 (Rigby, Jun. 10, 2004, “Methods and Apparatus for Transdermal Delivery of Abusable Drugs with a Deterrent Agent”), 20070104783 (Domb et al., May 10, 2007, “Double-Layered Absorbable Solid Compositions for the Topical Treatment of Oral Mucosal Disorders”), 20090130178 (Oronsky et al., May 21, 2009, “Formulation for Decreasing Tobacco, Alcohol, Drug or Food Consumption”), and 20120015021 (Mizrahi et al., Jan. 19, 2012, “Anti-Appetite Adhesive Compositions”).

### 6. Dental Appliance

This category of prior art includes dental appliances (or inserts) that contain and release a substance. In an example, the released substance can be a drug. It is not clear from the prior art how a dental appliance would selectively release a substance in response to food consumption. Also, the prior art does not disclose how a dental appliance could be selectively used to allow consumption of healthy food, but discourage consumption of unhealthy food. Also, the prior art does not disclose how a dental appliance could allow moderate consumption of certain foods, but limit excess consumption of those foods. Finally, if the appliance can be removed or must be refilled frequently, then this approach relies on voluntary compliance by the person.

Examples of prior art that appear to be best classified into this category include U.S. Pat. No. 5,194,003 (Garay et al., Mar. 16, 1993, “Removable Device for Delivering Beneficial Agents Orally”) and U.S. Pat. No. 8,181,655 (Bardach et al., May 22, 2012, “Therapeutic and Protective Dental Device Useful as an Intra-Oral Delivery System”); and U.S. Patent Applications 20080044797 (Bardach et al., Feb. 21, 2008, “Inserts for Use with Oral Appliances”) and 20120109051 (Harrell, May 3, 2012, “Devices, Methods, and Kits for Taste Modification and Controlling Food Intake”).

### 7. Oral or Nasal Spray

This category of prior art includes oral or nasal sprays (or a pulse of a gas) that contain a consumption-modifying substance. In an example, this substance can be absorbed into tissue for a systemic (pharmacologic) appetite-suppressant effect. In an example, this substance can be released into the person's oral cavity or nasal cavities for a localized anesthetic effect. In an example, this substance can mask or block the taste or smell of food. In order for this approach to work, the person must exercise consistent voluntary compliance in spraying the substance into their mouth or nose prior to consumption of (selected types of) food. However, if a person does not have enough willpower and discipline to avoid eating unhealthy food in the first place, then it is not clear that this person would have enough willpower and discipline to consistently spray something into their nose or mouth before each snack or meal.

Examples of prior art that appear to be best classified into this category include: U.S. Pat. No. 4,935,225 (Curtis et al., Jun. 19, 1990, “Appetite Suppresant Dentifrice”), U.S. Pat. No. 5,284,132 (Geier, Feb. 8, 1994, “Device for the Transnasal or Oral Administration of Drugs or the Like”), U.S. Pat. No. 5,456,677 (Spector, Oct. 10, 1995, “Method for Oral Spray Administration of Caffeine”), U.S. Pat. No. 6,715,485 (Djupesland, Apr. 6, 2004, “Nasal Delivery Device”), and U.S. Pat. No. 7,935,065 (Martin et al., May 3, 2011, “Oral Device”); and U.S. Patent Application 20050037031 (Jackson, Feb. 17, 2005, “Methods for Diet and Weight Control by Altering the Senses of Smell and Taste”).

### 8. Limiting Mouth Capacity or Function

This category of prior art includes devices and methods that limit mouth capacity or function so that a person eats less. In an example, a bulky device can be attached within a person's oral cavity to reduce the size of the cavity so that a person eats less with each mouthful. This assumes that the person will not simply eat more mouthfuls to compensate. In an example, a device can be attached to the person's mouth to create resistance to chewing motion so that eating takes more work and the person eats less. In an example, a device can block consumption of solid food. This assumes that blocking solid food is an effective way to modify a person's diet to manage their weight. In an example, a device can physically cover or shield a person's tongue and taste buds so that they eat less. This assumes that such a device will be tolerated and will not be removed.

It is not clear from the prior art how such devices could be selectively used to allow consumption of healthy food, but discourage consumption of unhealthy food. Also, the prior art does not disclose how such a device could allow moderate consumption of certain foods but limit excess consumption of those foods. Also, if the device is removable, then it requires consistent voluntary compliance by the person in order to be effective.

Examples of prior art that appear to be best classified into this category include: U.S. Pat. No. 3,818,906 (Stubbs, Jun. 25, 1974, “Apparatus for Controlling Eating and Smoking Habits”), U.S. Pat. No. 4,471,771 (Brown; Steven et al.; Sep. 18, 1984, “Oral Weight Control Device”), U.S. Pat. No. 4,738,259 (Brown et al., Apr. 19, 1988, “Dental Appliance for Weight Control”), U.S. Pat. No. 5,924,422 (Gustafson, Jul. 20, 1999, “Oral Device to Aid Weight Control”), U.S. Pat. No. 5,979,449 (Steer, Nov. 9, 1999, “Oral Appliance Device and Method for use Thereof for Appetite Suppression”), U.S. Pat. No. 6,422,243 (Daram, Jul. 23, 2002, “Taste Bud Shield and Method of Using Same”), and U.S. Pat. No. 8,230,865 (Shalon, Jul. 31, 2012, “Palatal Implant”); and U.S. Patent Applications 20030059737 (Hall, Mar. 27, 2003, “Obesity Treatment Aid”) and 20050287495 (Longley, Dec. 29, 2005, “Dental Appliance for Weight Management”).

### 9. Non-Consumption-Related Mouth Implant

This category of prior art includes devices that are implanted or attached within the mouth, but are not created for the purpose of modifying food consumption. They are included in this review of the prior art because they show examples of how implanted or attached devices can be tolerated within the human mouth. Examples of devices in this category include hearing aids and tracking devices that are attached within the oral cavity.

Examples of prior art that appear to be best classified into this category include: U.S. Pat. No. 4,244,373 (Nachman, Jan. 13, 1981, “Electrical Stimulation Dental Device”), U.S. Pat. No. 6,239,705 (Glen, May 29, 2001, “Intra Oral Electronic Tracking Device”), U.S. Pat. No. 7,724,911 (Menzel et al., May 25, 2010, “Actuator Systems for Oral-Based Appliances”), and U.S. Pat. No. 8,170,242 (Menzel et al., May 1, 2012, “Actuator Systems for Oral-Based Appliances”).

### 10. Implantable Drug Pump

This category of prior art includes implantable drug pumps that are used to achieve a (systemic) consumption-modifying effect. Not all implantable drug pumps are reviewed here, only those which appear to be most relevant to modification of food consumption. In an example, an implantable pump may pump a drug into the person's brain. In an example, an implantable pump can pump a drug into a location along the person's digestive tract. It is not clear from the prior art how such devices could be selectively used to allow consumption of healthy food, but discourage consumption of unhealthy food. Also, the prior art does not disclose how such devices could be used to allow moderate consumption, but limit excess consumption, of certain foods.

Examples of prior art that appear to be best classified into this category include: U.S. Pat. No. 5,643,207 (Rise, Jul. 1, 1997, “Implantable Techniques for Infusing a Therapeutic Agent with Endogenous Bodily Fluid”), U.S. Pat. No. 5,782,798 (Rise, Jul. 21, 1998, “Techniques for Treating Eating Disorders by Brain Stimulation and Drug Infusion”), U.S. Pat. No. 6,270,455 (Brown, Aug. 7, 2001, “Networked System for Interactive Communications and Remote Monitoring of Drug Delivery”), U.S. Pat. No. 6,334,073 (Levine, Dec. 25, 2001, “Internal Monitoring and Behavior Control System”), U.S. Pat. No. 6,950,707 (Whitehurst, Sep. 27, 2005, “Systems and Methods for Treatment of Obesity and Eating Disorders by Electrical Brain Stimulation and/or Drug Infusion”), U.S. Pat. No. 7,043,295 (Starkebaum, May 9, 2006, “Methods and Apparatus for Delivering a Drug Influencing Appetite for Treatment of Eating Disorders”), U.S. Pat. No. 7,108,680 (Rohr et al., Sep. 19, 2006, “Closed-Loop Drug Delivery System”), U.S. Pat. No. 7,790,671 (Stojanovic-Susulic et al., Sep. 7, 2010, “Implantable Pump for Protein Delivery for Obesity Control by Drug Infusion into the Brain”), and U.S. Pat. No. 8,252,744 (Stojanovic-Susulic et al., Aug. 28, 2012, “Implantable Pump for Protein Delivery for Obesity Control by Drug Infusion into the Brain”); and U.S. Patent Applications 20030171711 (Rohr et al., Sep. 11, 2003, “Closed-Loop Drug Delivery System”) and 20050038415 (Rohr et al., Feb. 17, 2005, “Method and Apparatus for the Treatment of Obesity”); EP 1504778, “Implantable Pump for the Treatment of Obesity”), WO 2002085428, “Implantable Osmotic Pump”), WO 2003004034, “Method for Inducing Analgesia Comprising Administration Alternatively of an Opioid Receptor Agonist . . . ”).

### 11. Implantable Glucose Sensor

This category of prior art includes sensors which monitor blood glucose level. This category is an example of a sensor which can measure the amount of a nutrient or other consumption-related substance within the body. It is not clear from the prior art how such a device could automatically modify a person's eating patterns. If a person does not have enough willpower and discipline to avoid eating a type of food that they already know is high in sugar, then it is not clear that this person would modify their eating habits based on additional information about the food's high-sugar content. This approach in the prior art appears to rely on voluntary compliance by the person in order to change their eating habits.

Examples of prior art that appear to be best classified into this category include: U.S. Pat. No. 5,497,772 (Schulman et al., Mar. 12, 1996, “Glucose Monitoring System”), U.S. Pat. No. 7,974,672 (Shults et al., Jul. 5, 2011, “Device and Method for Determining Analyte Levels”), and U.S. Pat. No. 8,236,242 (Drucker et al., Aug. 7, 2012, “Blood Glucose Tracking Apparatus and Methods”).

### 12. Sound Sensor to Detect Consumption

This category of prior art includes sound sensors which detect food consumption. In an example, such a sensor can detect chewing or swallowing sounds that indicate that a person is eating food. It is not clear from such prior art how well, if at all, such sensors would be able to differentiate between consumption of unhealthy vs. healthy food. Also, it is not clear from the prior art how such sensors could be used to automatically and selectively modify a person's eating habits. Such a sensor might produce data, but would such data automatically change eating behavior?

Examples of prior art that appear to be best classified into this category include: U.S. Pat. No. 7,914,468 (Shalon et al., Mar. 29, 2011, “Systems and Methods for Monitoring and Modifying Behavior”); and U.S. Patent Applications 20060064037 (Shalon et al., Mar. 23, 2006, “Systems and Methods for Monitoring and Modifying Behavior”) and 20110125063 (Shalon et al., May 26, 2011, “Systems and Methods for Monitoring and Modifying Behavior”).

### 13. Digestive Tract Sensor

This category of prior art includes sensors which monitor activity of the stomach, esophagus, or other portions of the digestive tract past the person's mouth. In an example, sensors in this category can monitor the electrical activity the muscles or nerves associated with the stomach. In an example, sensors in this category can measure pressure exerted by expansion of stomach walls.

Although some examples of such prior art can generally differentiate between consumption of solid food vs. liquid food, it is not clear how well (if at all) such sensors would be able to differentiate between consumption of unhealthy food vs. healthy food. Also, it is not clear from the prior art how such sensors could be used to automatically and selectively modify a person's eating habits in real time. Once food has already reached the stomach (where it is first detected by such sensors), one has to interrupt absorption or cause emesis to avoid having such food become fully digested—both of which have limitations.

Examples of prior art that appear to be best classified into this category include: U.S. Pat. No. 4,823,808 (Clegg et al., Apr. 25, 1989, “Method for Control of Obesity Overweight and Eating Disorders”), U.S. Pat. No. 5,292,344 (Douglas, Mar. 8, 1994, “Percutaneously Placed Electrical Gastrointestinal Pacemaker Stimulatory System, Sensing System, and PH Monitoring . . . ”), U.S. Pat. No. 5,795,304 (Sun et al., Aug. 18, 1998, “System and Method for Analyzing Electrogastrophic Signal”), U.S. Pat. No. 6,735,477 (Levine, May 11, 2004, “Internal Monitoring System with Detection of Food Intake”), U.S. Pat. No. 7,076,305 (Imran et al., Jul. 11, 2006, “Gastric Device and Instrument System and Method”), U.S. Pat. No. 7,330,753 (Policker et al., Feb. 12, 2008, “Analysis of Eating Habits”), U.S. Pat. No. 7,437,195 (Policker et al., Oct. 14, 2008, “Regulation of Eating Habits”), U.S. Pat. No. 7,702,394 (Imran, Apr. 20, 2010, “Responsive Gastric Stimulator”), U.S. Pat. No. 7,832,407 (Gertner, Nov. 16, 2010, “Obesity Treatment Systems”), U.S. Pat. No. 7,840,269 (Policker et al., Nov. 23, 2010, “Analysis of Eating Habits”), and U.S. Pat. No. 8,239,027 (Imran, Aug. 7, 2012, “Responsive Gastric Stimulator”).

Examples of prior art that appear to be best classified into this category also include: U.S. Patent Applications 20030009202 (Levine, Jan. 9, 2003, “Internal Monitoring System with Detection of Food Intake”), 20040147816 (Policker et al., Jul. 29, 2004, “Analysis of Eating Habits”), 20040162595 (Foley, Aug. 19, 2004, “Method and Apparatus for Intentional Impairment of Gastric Motility and/or Efficiency by Triggered Electrical Stimulation . . . ”), 20050096514 (Starkebaum, May 5, 2005, “Gastric Activity Notification”), 20050222638 (Foley et al., Oct. 6, 2005, “Sensor Based Gastrointestinal Electrical Stimulation for the Treatment of Obesity or Motility Disorders”), 20060089571 (Gertner, Apr. 27, 2006, “Obesity Treatment Systems”), 20060173238 (Starkebaum, Aug. 3, 2006, “Dynamically Controlled Gastric Occlusion Device”), 20060199997 (Hassler et al., Sep. 7, 2006, “Monitoring of a Food Intake Restriction Device”), 20070255334 (Keimel et al., Nov. 1, 2007, “Energy Balance Therapy for Obesity Management”), 20070299320 (Policker et al., Dec. 27, 2007, “Analysis of Eating Habits”), 20090118797 (Kliger et al., May 7, 2009, “Monitoring, Analysis, and Regulation of Eating Habits”), and 20090240194 (Keimel et al., Sep. 24, 2009, “Energy Balance Therapy for Obesity Management”).

Examples of prior art that appear to be best classified into this category also include: U.S. Patent Applications 20090281449 (Thrower et al., Nov. 12, 2009, “Optimization of Thresholds for Eating Detection”), 20100076345 (Soffer et al., Mar. 25, 2010, “Method, Device and System for Automatic Detection of Eating and Drinking”), 20100094374 (Imran, Apr. 15, 2010, “Responsive Gastric Stimulator”), 20100152532 (Marcotte, Jun. 17, 2010, “Gastric Band System with Esophageal Sensor”), 20100160745 (Hills et al., Jun. 24, 2010, “Detection of Food or Drink Consumption in Order to Control Therapy or Provide Diagnostics”), 20100305468 (Policker et al., Dec. 2, 2010, “Analysis and Regulation of Food Intake”), 20100324432 (Bjorling et al., Dec. 23, 2010, “Method and Device to Detect Eating to Control Artificial Gastric Stimulation”), 20110034760 (Brynelsen et al., Feb. 10, 2011, “Feedback Systems and Methods to Enhance Obstructive and Other Obesity Treatments”), 20110066207 (Imran, Mar. 17, 2011, “Responsive Gastric Stimulator”), 20120101874 (Ben-Haim et al., Apr. 26, 2012, “Charger With Data Transfer Capabilities”), 20120116182 (Wong et al., May 10, 2012, “Feedback Systems and Methods to Enhance Obstructive and Other Obesity Treatments . . . ”), and 20120214140 (Brynelsen et al., Aug. 23, 2012, “Feedback Systems and Methods for Communicating Diagnostic and/or Treatment Signals . . . ”); WO 2002082968, “Analysis of Eating Habits”).

14. Restricting Food Volume or Absorption

This category of prior art includes devices and methods that restrict the volume of food passing through the digestive tract and/or reduce absorption of food along the digestive tract. This category includes external bands around the stomach that restrict the rate at which food can enter the stomach. This category also includes various devices, such as gastric balloons, that take up space within the stomach to reduce the volume of food which the stomach can hold. This category also includes internal sleeves and other devices that keep food separated from the digestive surfaces along some portion of the digestive tract.

It is not clear from the prior art how devices that are based on restricting food volume or absorption can be used: to differentiate between unhealthy foods vs. healthy foods; or to modify eating habits. Accordingly, such devices can have the undesirable side effect of reducing absorption of necessary nutrients. Also, such devices still rely on voluntary compliance to some extent because people with such implanted devices can still consume unhealthy foods, or excess quantities of food, by consuming food at a slower rate or in a more-liquid form. Someone with a gastric restriction device can still consume a half-gallon of ice cream during a nocturnal eating binge; it just takes longer.

Examples of prior art that appear to be best classified into this category include: U.S. Pat. No. 4,133,315 (Berman et al., Jan. 9, 1979, “Method and Apparatus for Reducing Obesity”), U.S. Pat. No. 4,416,267 (Garren et al., Nov. 22, 1983, “Method and Apparatus for Treating Obesity”), U.S. Pat. No. 4,592,339 (Kuzmak et al., Jun. 3, 1986, “Gastric Banding Device”), U.S. Pat. No. 5,074,868 (Kuzmak, Dec. 24, 1991, “Reversible Stoma-Adjustable Gastric Band”), U.S. Pat. No. 5,226,429 (Kuzmak, Jul. 13, 1993, “Laparoscopic Gastric Band and Method”), U.S. Pat. No. 5,234,454 (Bangs, Aug. 10, 1993, “Percutaneous Intragastric Balloon Catheter and Method for Controlling Body Weight Therewith”), U.S. Pat. No. 5,449,368 (Kuzmak, Sep. 12, 1995, “Laparoscopic Adjustable Gastric Banding Device and Method for Implantation and Removal Thereof”), U.S. Pat. No. 5,601,604 (Vincent, Feb. 11, 1997, “Universal Gastric Band”), U.S. Pat. No. 5,868,141 (Ellias, Feb. 9, 1999, “Endoscopic Stomach Insert for Treating Obesity and Method for Use”), U.S. Pat. No. 6,067,991 (Forsell, May 30, 2000, “Mechanical Food Intake Restriction Device”), U.S. Pat. No. 7,963,907 (Gertner, Jun. 21, 2011, “Closed Loop Gastric Restriction Devices and Methods”), U.S. Pat. No. 8,016,744 (Dlugos et al., Sep. 13, 2011, “External Pressure-Based Gastric Band Adjustment System and Method”), and U.S. Pat. No. 8,016,745 (Hassler et al., Sep. 13, 2011, “Monitoring of a Food Intake Restriction Device”).

Examples of prior art that appear to be best classified into this category also include: U.S. Patent Applications 20060264699 (Gertner, Nov. 23, 2006, “Extragastric Minimally Invasive Methods and Devices to Treat Obesity”), 20070293885 (Binmoeller, Dec. 20, 2007, “Methods and Devices to Curb Appetite and/or to Reduce Food Intake”), 20080065168 (Bitton et al., Mar. 13, 2008, “Ingestible Capsule for Appetite Regulation”), 20090306462 (Lechner, Dec. 10, 2009, “System for Controlling a Controllable Stomach Band”), 20100152764 (Merkle, Jun. 17, 2010, “Device for Treating Obesity”), 20100312049 (Forsell, Dec. 9, 2010, “Apparatus for Treating Obesity”), 20100312050 (Forsell, Dec. 9, 2010, “Method and Instrument for Treating Obesity”), 20100312147 (Gertner, Dec. 9, 2010, “Obesity Treatment Systems”), 20100331616 (Forsell, Dec. 30, 2010, “Method and Instrument for Treating Obesity”), 20100331617 (Forsell, Dec. 30, 2010, “Device, System and Method for Treating Obesity”), 20100332000 (Forsell, Dec. 30, 2010, “Device for Treating Obesity”), 20110172693 (Forsell, Jul. 14, 2011, “Apparatus and Method for Treating Obesity”), 20110196411 (Forsell, Aug. 11, 2011, “Apparatus for Treating Obesity”), and 20120029550 (Forsell, Feb. 2, 2012, “Obesity Treatment”), 20120041463 (Forsell, Feb. 16, 2012, “Obesity Treatment”).

### 15. Applying Electricity to Digestive Tract Organs

This category of prior art includes devices and methods wherein electricity is applied to the stomach, or to other organs along the digestive tract, or to the nerves which are connected to those organs. In an example, devices in this category can use electric pulses to stimulate the stomach, other digestive tract organs, or the nerves associated with those organs. In an example, devices in this category use electric pulses to block nerve transmissions required for the normal functioning of the stomach or other digestive tract organs. It is not clear how well (if at all) such devices and methods would be able to differentiate between consumption of unhealthy food vs. healthy food. Also, it is not clear how such prior art could be used to automatically and selectively modify a person's eating habits in real time.

Examples of prior art that appear to be best classified into this category include U.S. Pat. No. 3,411,507 (Wingrove Nov. 19, 1968, “Method of Gastrointestinal Stimulation with Electrical Pulses”), U.S. Pat. No. 5,188,104 (Wernicke et al., Feb. 23, 1993, “Treatment of Eating Disorders by Nerve Stimulation”), U.S. Pat. No. 5,263,480 (Wernicke et al., Nov. 23, 1993, “Treatment of Eating Disorders by Nerve Stimulation”), U.S. Pat. No. 5,423,872 (Cigaina, Jun. 13, 1995, “Process and Device for Treating Obesity and Syndromes Related to Motor Disorders of the Stomach of a Patient”), U.S. Pat. No. 5,540,730 (Terry et al., Jul. 30, 1996, “Treatment of Motility Disorders by Nerve Stimulation”), U.S. Pat. No. 5,690,691 (Chen et al., Nov. 25, 1997, “Gastro-Intestinal Pacemaker Having Phased Multi-Point Stimulation”), U.S. Pat. No. 5,716,385 (Mittal et al., Feb. 10, 1998, “Crural Diaphragm Pacemaker and Method for Treating Esophageal Reflux Disease”), U.S. Pat. No. 5,836,994 (Bourgeois, Nov. 17, 1998, “Method and Apparatus for Electrical Stimulation of the Gastrointestinal Tract”), U.S. Pat. No. 5,861,014 (Familoni, Jan. 19, 1999, “Method and Apparatus for Sensing a Stimulating Gastrointestinal Tract On-Demand”), U.S. Pat. No. 5,891,185 (Freed et al., Apr. 6, 1999, “Method and Apparatus for Treating Oropharyngeal Disorders with Electrical Stimulation”), U.S. Pat. No. 5,995,872 (Bourgeois, Nov. 30, 1999, “Method and Apparatus for Electrical Stimulation of the Gastrointestinal Tract”), U.S. Pat. No. 6,091,992 (Bourgeois et al., Jul. 18, 2000, “Method and Apparatus for Electrical Stimulation of the Gastrointestinal Tract”), and U.S. Pat. No. 6,104,955 (Bourgeois, Aug. 15, 2000, “Method and Apparatus for Electrical Stimulation of the Gastrointestinal Tract”),

Examples of prior art that appear to be best classified into this category include U.S. Pat. No. 6,115,635 (Bourgeois, Sep. 5, 2000, “Method and Apparatus for Electrical Stimulation of the Gastrointestinal Tract”), U.S. Pat. No. 6,185,452 (Schulman et al., Feb. 6, 2001, “Battery-Powered Patient Implantable Device”), U.S. Pat. No. 6,327,503 (Familoni, Dec. 4, 2001, “Method and Apparatus for Sensing and Stimulating Gastrointestinal Tract On-Demand”), U.S. Pat. No. 6,535,764 (Imran et al., Mar. 18, 2003, “Gastric Treatment and Diagnosis Device and Method”), U.S. Pat. No. 6,591,137 (Fischell et al., Jul. 8, 2003, “Implantable Neuromuscular Stimulator for the Treatment of Gastrointestinal Disorders”), U.S. Pat. No. 6,600,953 (Flesler et al., Jul. 29, 2003, “Acute and Chronic Electrical Signal Therapy for Obesity”), U.S. Pat. No. 6,609,025 (Barrett et al., Aug. 19, 2003, “Treatment of Obesity by Bilateral Sub-Diaphragmatic Nerve Stimulation”), U.S. Pat. No. 6,993,391 (Flesler et al., Jan. 31, 2006, “Acute and Chronic Electrical Signal Therapy for Obesity”), U.S. Pat. No. 7,054,690 (Imran, May 30, 2006, “Gastrointestinal Stimulation Device”), U.S. Pat. No. 7,120,498 (Imran et al., Oct. 10, 2006, “Method and Device for Securing a Functional Device to a Stomach”), U.S. Pat. No. 7,167,750 (Knudson et al., Jan. 23, 2007, “Obesity Treatment with Electrically Induced Vagal Down Regulation”), U.S. Pat. No. 7,299,091 (Barrett et al., Nov. 20, 2007, “Treatment of Obesity by Bilateral Vagus Nerve Stimulation”), U.S. Pat. No. 7,430,450 (Imran, Sep. 30, 2008, “Device and Method for Treating Obesity”), U.S. Pat. No. 7,509,174 (Imran et al., Mar. 24, 2009, “Gastric Treatment/Diagnosis Device and Attachment Device and Method”), U.S. Pat. No. 7,512,442 (Flesler et al., Mar. 31, 2009, “Acute and Chronic Electrical Signal Therapy For Obesity”), U.S. Pat. No. 7,729,771 (Knudson et al., Jun. 1, 2010, “Nerve Stimulation and Blocking for Treatment of Gastrointestinal Disorders”), U.S. Pat. No. 7,979,127 (Imran, Jul. 12, 2011, “Digestive Organ Retention Device”), U.S. Pat. No. 7,986,995 (Knudson et al., Jul. 26, 2011, “Bulimia Treatment”), and U.S. Pat. No. 8,010,204 (Knudson et al., Aug. 30, 2011, “Nerve Blocking for Treatment of Gastrointestinal Disorders”).

Examples of prior art that appear to be best classified into this category also include: U.S. Patent Applications 20040059393 (Policker et al., Mar. 25, 2004, “Regulation of Eating Habits”), 20040167583 (Knudson et al., Aug. 26, 2004, “Electrode Band Apparatus and Method”), 20050038484 (Knudson et al., Feb. 17, 2005, “Controlled Vagal Blockage Therapy”), 20050131485 (Knudson et al., Jun. 16, 2005, “High Frequency Vagal Blockage Therapy”), 20060173508 (Stone et al., Aug. 3, 2006, “Method and System for Treatment of Eating Disorders by Means of Neuro-Electrical Coded Signals”), 20060206169 (Schuler, Sep. 14, 2006, “Method and System for Modulating Eating Behavior by Means of Neuro-Electrical Coded Signals”), 20060235487 (Meyer et al., Oct. 19, 2006, “Method and System for Treatment of Eating Disorders by Means of Neuro-Electrical Coded Signals”), 20070027493 (Ben-Haim et al., Feb. 1, 2007, “Gastrointestinal Methods and Apparatus for Use in Treating Disorders and Controlling Blood Sugar”), 20070135846 (Knudson et al., Jun. 14, 2007, “Vagal Obesity Treatment”), 20080021512 (Knudson et al., Jan. 24, 2008, “Nerve Stimulation and Blocking for Treatment of Gastrointestinal Disorders”), 20090018605 (Imran et al., Jan. 15, 2009, “Gastric Treatment/Diagnosis Device and Attachment Device and Method”), 20090018606 (Sparks et al., Jan. 15, 2009, “Methods and Devices for Stimulation of an Organ with the Use of a Transectionally Placed Guide Wire”), and 20090030474 (Brynelsen et al., Jan. 29, 2009, “Sensor Driven Gastric Stimulation for Patient Management”).

Examples of prior art that appear to be best classified into this category also include: U.S. Patent Applications 20090030475 (Brynelsen et al., Jan. 29, 2009, “Gastric Stimulation Systems and Methods Utilizing a Transgastric Probe”), 20100049274 (Cholette, Feb. 25, 2010, “Detection of Feeding Intent for Use in Treatment of Eating Disorders”), 20100087706 (Syed et al., Apr. 8, 2010, “Lead Access”), 20100094375 (Donders et al., Apr. 15, 2010, “Neural Electrode Treatment”), 20100168815 (Knudson et al., Jul. 1, 2010, “Nerve Stimulation and Blocking for Treatment of Gastrointestinal Disorders”), 20100234917 (Imran, Sep. 16, 2010, “Digestive Organ Retention Device”), 20100286745 (Imran, Nov. 11, 2010, “Radially Expandable Gastrointestinal Stimulation Device”), 20100305656 (Imran et al., Dec. 2, 2010, “Gastric Simulation Anchor and Method”), 20110034967 (Chen et al., Feb. 10, 2011, “Gastrointestinal Electrical Stimulation”), 20110034968 (Knudson et al., Feb. 10, 2011, “Controlled Vagal Blockage Therapy”), 20110166582 (Syed et al., Jul. 7, 2011, “Endoscopic Device Delivery System”), 20110270344 (Knudson et al., Nov. 3, 2011, “Bulimia Treatment”), 20110282411 (Knudson et al., Nov. 17, 2011, “Nerve Stimulation and Blocking for Treatment of Gastrointestinal Disorders”), 20110307023 (Tweden et al., Dec. 15, 2011, “Neural Modulation Devices and Methods”), and 20120041509 (Knudson et al., Feb. 16, 2012, “Controlled Vagal Blockage Therapy”).

### 16. Stimulating Salivation

This category of prior art includes devices and methods for stimulating salivation in a person's mouth. In some respects, this is quite different than the devices and methods in prior categories that are intended to reduce food consumption. Most devices and methods in this category are focused on increasing, not decreasing, food consumption. However, this category is included for completeness because some of these devices are intended to modify the early stages of food digestion within a person's mouth, which is relevant to the present invention. In an example, devices in this category can apply electrical stimulation to the mouth to increase salivation. In an example, devices in this category can release a salivation-stimulating substance. Prior art in this category does not appear to disclose selective reduction of consumption of unhealthy foods.

Examples of prior art that appear to be best classified into this category include: U.S. Pat. No. 4,519,400 (Brenman et al., May 28, 1985, “Method for Stimulating Salivation”), U.S. Pat. No. 4,637,405 (Brenman et al., Jan. 20, 1987, “Apparatus for Stimulating Salivation”), U.S. Pat. No. 6,230,052 (Wolff et al., May 8, 2001, “Device and Method for Stimulating Salivation”), U.S. Pat. No. 7,041,311 (Grainger et al., May 9, 2006, “Preparation for Saliva Flow”), and U.S. Pat. No. 7,477,947 (Pines et al., Jan. 13, 2009, “System and Method for Electrical Stimulation of Salivation”); and U.S. Patent Application 20070077300 (Wynn et al., Apr. 5, 2007, “Oral Compositions Containing a Salivation Inducing Agent”).

### 17. Diversion of Enzyme Secretion

This category of prior art includes devices and methods that divert, or otherwise modify, the flow of digestive enzymes from body organs into the digestive tract. It is not clear from the prior art how well (if at all) such devices and methods can differentiate between consumption of unhealthy food vs. healthy food. Also, it is not clear how such prior art could be used to automatically and selectively modify a person's eating habits.

Examples of prior art that appear to be best classified into this category include U.S. Pat. No. 7,833,279 (Knudson et al., Nov. 16, 2010, “Pancreatic Exocrine Secretion Diversion Apparatus and Method”); and U.S. Patent Applications 20060106332 (Knudson et al., May 18, 2006, “Pancreatic Exocrine Secretion Diversion Apparatus and Method”) and 20110021968 (Knudson et al., Jan. 27, 2011, “Pancreatic Exocrine Secretion Diversion Apparatus and Method”).

### 18. General Feedback and Support Systems for Energy Balance

This category of prior art includes a wide variety of relatively-general systems, devices and methods that are intended to provide a person with support and feedback concerning their energy balance and weight management. In various examples, systems, devices, and methods in this category can involve: general feedback and behavior modification concerning diet and exercise patterns; broadly-defined use of general types of sensors for energy balance and weight management; interactive communication between people and healthcare providers, or between people and social support networks; internet websites that provide online support for energy balance and weight management; and general meal planning systems and methods. Much of the prior art in this category can be very useful, but is very general compared to the specificity of this present invention. However, this general category is included in this review in order to be thorough.

Examples of prior art that appear to be best classified into this category include: U.S. Pat. No. 4,951,197 (Mellinger, Aug. 21, 1990, “Weight Loss Management System”), U.S. Pat. No. 5,673,691 (Abrams et al., Oct. 7, 1997, “Apparatus to Control Diet and Weight Using Human Behavior Modification Techniques”), U.S. Pat. No. 5,720,771 (Snell, Feb. 24, 1998, “Method and Apparatus for Monitoring Physiological Data from an Implantable Medical Device”), U.S. Pat. No. 6,154,676 (Levine, Nov. 28, 2000, “Internal Monitoring and Behavior Control System”), U.S. Pat. No. 6,334,073 (Levine, Dec. 25, 2001, “Internal Monitoring and Behavior Control System”), U.S. Pat. No. 6,735,477 (Levine, May 11, 2004, “Internal Monitoring System with Detection of Food Intake”), U.S. Pat. No. 6,735,479 (Fabian et al., May 11, 2004, “Lifestyle Management System”), U.S. Pat. No. 7,247,023 (Peplinski et al., Jul. 24, 2007, “System and method for monitoring weight and nutrition”), U.S. Pat. No. 7,558,629 (Keimel et al., Jul. 7, 2009, “Energy Balance Therapy for Obesity Management”), U.S. Pat. No. 7,882,150 (Badyal, Feb. 1, 2011, “Health Advisor”), and U.S. Pat. No. 8,135,470 (Keimel et al., Mar. 13, 2012, “Energy Balance Therapy for Obesity Management”).

Examples of prior art that appear to be best classified into this category also include: U.S. Patent Applications 20030009202 (Levine, Jan. 9, 2003, “Internal Monitoring System with Detection of Food Intake”), 20050096637 (Heruth, May 5, 2005, “Sensing Food Intake”), 20050113649 (Bergantino, May 26, 2005, “Method and Apparatus for Managing a User's Health”), 20050113650 (Pacione et al., May 26, 2005, “System for Monitoring and Managing Body Weight and Other Physiological Conditions Including Iterative . . . ”), 20060015016 (Thornton, Jan. 19, 2006, “Caloric Balance Weight Control System and Methods of Making and Using Same”), 20060020298 (Camilleri et al., Jan. 26, 2006, “Systems and Methods for Curbing Appetite”), 20060122468 (Tavor, Jun. 8, 2006, “Nutritional Counseling Method and Server”), 20070021979 (Cosentino et al., Jan. 25, 2007, “Multiuser Wellness Parameter Monitoring System”), 20070042058 (George et al., Feb. 22, 2007, “System and method for monitoring weight and nutrition”), 20080221644 (Vallapureddy et al., Sep. 11, 2008, “Remote Monitoring and Control of Implantable Devices”), 20110087076 (Brynelsen et al., Apr. 14, 2011, “Feedback Systems and Methods for Communicating Diagnostic and/or Treatment Signals to Enhance Obesity Treatments”), 20110251495 (Province et al., Oct. 13, 2011, “Diagnostic Sensors and/or Treatments for Gastrointestinal Stimulation or Monitoring Devices”), and 20120065706 (Vallapureddy et al., Mar. 15, 2012, “Remote Monitoring and Control of Implantable Devices”).

### 19. Methods to Identify Compounds or Substances

This category of prior art includes methods for identification and/or classification of specific compounds or substances that are intended to affect food consumption. Such prior art does not appear to disclose or predict the automatic and selective taste and/or smell modification functions of this present invention. However, methods for identification of specific compounds or substances for taste and/or smell modification can be useful in identifying which substances are used for taste and/or smell modification in this present invention.

Examples of prior art that appear to be best classified into this category include: U.S. Pat. No. 5,858,967 (Weigle et al., Jan. 12, 1999, “Appetite Supression Factor and Related Methods”), U.S. Pat. No. 7,241,880 (Adler et al., Jul. 10, 2007, “T1R Taste Receptors and Genes Encoding Same”), and U.S. Pat. No. 8,067,185 (Zoller et al., Nov. 29, 2011, “Methods of Quantifying Taste of Compounds for Food or Beverages”).

### 20. Specific Compounds or Substances

This category of prior art includes specific compounds or substances that are intended to affect food consumption. In various examples, such specific substances and compounds can include naturally-occurring substances (such as plant extracts) that are used with the intent of modifying food consumption. In various examples, such specific substances and compounds can include manufactured substances that are used with the intent of modifying food consumption. There is extensive prior art in this category as the search continues for an elusive “magic pill” that might safely and significantly reduce food consumption. Most, if not all, of the prior art in this category relies on consistent voluntary compliance for ingestion of the substance or compound by the person. Also, prior art in this category does not appear to disclose how to automatically and selectively discourage consumption of unhealthy food, while allowing unmitigated consumption of healthy food.

Examples of prior art that appear to be best classified into this category include: U.S. Pat. No. 4,159,347 (Yoshida et al., Jun. 26, 1979, “Flavoring with Cyclic Acetals of 2-Methyl-2-Pentenal”), U.S. Pat. No. 4,210,637 (Wurtman et al., Jul. 1, 1980, “Composition and Method for Suppressing Appetite for Calories as Carbohydrates”), U.S. Pat. No. 4,491,578 (Peikin, Jan. 1, 1985, “Method of Stimulating Satiety in Mammals”), U.S. Pat. No. 4,497,798 (Lambert, Feb. 5, 1985, “Appetite Suppressant”), U.S. Pat. No. 4,689,235 (Barnes et al., Aug. 25, 1987, “Encapsulation Matrix Composition and Encapsulate Containing Same”), U.S. Pat. No. 4,740,365 (Yukimatsu et al., Apr. 26, 1988, “Sustained-Release Preparation Applicable to Mucous Membrane in Oral Cavity”), U.S. Pat. No. 5,013,716 (Cherukuri et al., May 7, 1991, “Unpleasant Taste Masking Compositions and Methods for Preparing Same”), U.S. Pat. No. 5,290,808 (Sofia, Mar. 1, 1994, “Method to Control the Intake of Food”), U.S. Pat. No. 5,405,641 (Kurihara et al., Apr. 11, 1995, “Taste-Modification Composition and Method for Stabilizing Taste-Modifier”), U.S. Pat. No. 5,472,685 (Gaffar, Dec. 5, 1995, “Antiplaque Oral Compositions”), U.S. Pat. No. 5,605,698 (Ueno, Feb. 25, 1997, “Oral Composition”), U.S. Pat. No. 6,123,980 (Pearson et al., Sep. 26, 2000, “Preparing Granulated Sugar Blends and Products”), U.S. Pat. No. 6,207,638 (Portman, Mar. 27, 2001, “Nutritional Intervention Composition for Enhancing and Extending Satiety”), U.S. Pat. No. 6,224,873 (Jones, May 1, 2001, “Regulation of Appetite Body Weight and Athletic Function with Materials Derived from Citrus Varieties”), and U.S. Pat. No. 6,235,274 (Lou et al., May 22, 2001, “Microparticles Which Controllably Release Olfactorily Active Substances Methods of Using Same and Processes for Preparing Same”).

Examples of prior art that appear to be best classified into this category also include: U.S. Pat. No. 6,319,523 (Zhou, Nov. 20, 2001, “Composition and Method for Inhibiting Oral Bacteria”), U.S. Pat. No. 6,376,657 (Van Heerden et al., Apr. 23, 2002, “Pharmaceutical Compositions Having Appetite Suppressant Activity”), U.S. Pat. No. 6,413,545 (Alviar et al., Jul. 2, 2002, “Diet Composition and Method of Weight Management”), U.S. Pat. No. 6,610,277 (Zuckerman, Aug. 26, 2003, “Appetite Suppressant Toothpaste”), U.S. Pat. No. 6,861,405 (Desir et al., Mar. 1, 2005, “Compositions and Methods Relating to Glucose Metabolism, Weight Control, and Food Intake”), U.S. Pat. No. 6,942,848 (Nelson et al., Sep. 13, 2005, “Cyclodextrins in Dental Products”), U.S. Pat. No. 7,138,107 (Adams et al., Nov. 21, 2006, “Inhibition of Olfactory Neurosensory Function to Treat Eating Disorders and Obesity”), U.S. Pat. No. 7,229,658 (Inoue et al., Jun. 12, 2007, “Compositions Containing Sucralose and Application Thereof”), U.S. Pat. No. 7,276,229 (Baker et al., Oct. 2, 2007, “Oral Compositions”), U.S. Pat. No. 7,524,877 (Rosenfeld et al., Apr. 28, 2009, “Compounds for Use in Weight Loss and Appetite Suppression in Humans”), U.S. Pat. No. 7,541,356 (Rosenfeld et al., Jun. 2, 2009, “Compounds for Use in Weight Loss and Appetite Suppression in Humans”), U.S. Pat. No. 7,632,517 (Dugger et al., Dec. 15, 2009, “Buccal Polar and Non-Polar Spray Containing Zolpidem”), U.S. Pat. No. 7,851,005 (Bingley et al., Dec. 14, 2010, “Taste Potentiator Compositions and

Beverages Containing Same”), U.S. Pat. No. 7,851,006 (Bingley et al., Dec. 14, 2010, “Taste Potentiator Compositions and Beverages Containing Same”), U.S. Pat. No. 7,879,376 (Boghani et al., Feb. 1, 2011, “Taste Potentiator Compositions and Edible Confectionery and Chewing Gum Products Containing Same”), U.S. Pat. No. 8,217,001 (Cowley et al., Jul. 10, 2012, “Modification of Feeding Behavior”), and U.S. Pat. No. 8,236,285 (Dugger et al., Aug. 7, 2012, “Buccal, Polar and Non-Polar Spray Containing Zolpidem”).

Examples of prior art that appear to be best classified into this category also include: U.S. Patent Applications 20020187204 (Alviar et al., Dec. 12, 2002, “Diet Composition and Method of Weight Management”), 20030113310 (Van Laere et al., Jun. 19, 2003, “Method for the Treatment of Obesity, Overweight and Fluctuations in Blood Insuline and/or Glucose Levels”), 20040071801 (Edell et al., Apr. 15, 2004, “Herbal Formulation of Gymnema Sylvestre as a Dietary Aid”), 20040156920 (Kane, Aug. 12, 2004, “Extracts From Plant and Non-Plant Biomass and Uses Thereof”), 20040192760 (Whittle et al., Sep. 30, 2004, “Pharmaceutical Formulations”), 20040247702 (Rajendran et al., Dec. 9, 2004, “Caralluma Extract Products and Processes for Making the Same”), 20050053555 (Pederson, Mar. 10, 2005, “Appetite Control Compositions and Methods of Use”), 20060105068 (Fleischner, May 18, 2006, “Dietary Supplement Formulations Containing Hoodia Gordonii”), 20060193795 (Zuckerman, Aug. 31, 2006, “Appetite Suppressant Mouth Spray”), 20070104805 (Udell, May 10, 2007, “Compositions of Hoodia Gordonii and Pinolenic Acid Derivatives”), 20070196436 (Abrahamse et al., Aug. 23, 2007, “Process for Preparing an Edible Composition Comprising Steroidal Glycosides”), and 20080102143 (Freis et al., May 1, 2008, “Uses for the Extract of a Plant of the Family Asclepiadaceae”).

Examples of prior art that appear to be best classified into this category also include: U.S. Patent Applications 20080138447 (Riggins et al., Jun. 12, 2008, “Method for Administering Appetite Suppressant and Composition Thereof”), 20080152705 (Udell et al., Jun. 26, 2008, “Corosolic Acid Formulation and Its Application for Weight-Loss Management and Blood Sugar Balance”), 20080255093 (Tam et al., Oct. 16, 2008, “Compositions and Methods for Treating Obesity and Related Disorders”), 20100098783 (Sommerfeld et al., Apr. 22, 2010, “Appetite Suppressant Composition”), 20120115778 (Karsenty et al., May 10, 2012, “Methods of Suppressing Appetite by the Administration of Antagonists of the Serotonin HTR1a or HTR2b Receptors . . . ”), 20120157409 (Cherkassky, Jun. 21, 2012, “Appetite Suppressant Product and Method”), and 20120208748 (Chen et al., Aug. 16, 2012, “Peptide Compositions and Methods for Treating Patients”); EP 1685834, “Use of Pinolenic Acid for the Treatment Of Obesity”), EP 2072048, “Use of Pinolenic Acid for the Treatment Of Obesity”).

### 21. Miscellaneous

Lastly, this category of prior art includes a variety of devices and methods that may be generally relevant, but which resist neat categorization. Examples of prior art in this miscellaneous category include: devices that change a person's eating speed; necklaces with video cameras to monitor food intake; devices that a person can use to manually shock their tongue when they have a craving; manual scratch and sniff devices for addition control; having a person manual sniff something with a bad smell in order to reduce their appetite; methods for identifying olfactory cells; methods for slow release of antibiotics; devices to increase airflow through the nose to enhance the sense of smell; applying magnets in an effort to reduce a person's appetite; and applying electricity to improve hygiene. None of the prior art in this miscellaneous category appears to disclose or predict the automatic and selective taste and/or smell modification features of this present invention.

Examples of prior art that appear to be best classified into this category include U.S. Pat. No. 4,582,492 (Etter et al., Apr. 15, 1986, “Method for Behavior Modification Using Olfactory Stimuli”), U.S. Pat. No. 4,975,682 (Kerr et al., Dec. 4, 1990, “Meal Minder Device”), U.S. Pat. No. 5,792,210 (Wamubu et al., Aug. 11, 1998, “Electrical Tongue Stimulator and Method for Addiction Treatment”), U.S. Pat. No. 6,145,503 (Smith, Nov. 14, 2000, “Olfactory Activator”), U.S. Pat. No. 6,159,145 (Satoh, Dec. 12, 2000, “Appetite Adjusting Tool”), U.S. Pat. No. 6,218,358 (Firestein et al., Apr. 17, 2001, “Functional Expression of, and Assay for, Functional Cellular Receptors In Vivo”), U.S. Pat. No. 8,060,220 (Liebergesell et al., Nov. 15, 2011, “Promotion of Oral Hygiene and Treatment of Gingivitis Other Periodontal Problems and Oral Mal Odor . . . ”), and U.S. Pat. No. 8,233,954 (Kling et al., Jul. 31, 2012, “Mucosal Sensor for the Assessment of Tissue and Blood Constituents and Technique for Using the Same”); and U.S. Patent Applications 20040186528 (Ries et al., Sep. 23, 2004, “Subcutaneous Implantable Medical Devices with Anti-Microbial Agents for Chronic Release”), 20090012433 (Fernstrom et al., Jan. 8, 2009, “Method, Apparatus and System for Food Intake and Physical Activity Assessment”), and 20090197963 (Llewellyn, Aug. 6, 2009, “Method and Compositions for Suppressing Appetite or Treating Obesity”).

### SUMMARY OF THIS INVENTION

This invention is a device and method for automatic and selective modification of the taste and/or smell of one or more selected nutrient or food types as food is being digested within a person's mouth. This invention can comprise: a taste and/or smell modifying substance; a reservoir for this substance that is implanted within the person's body; and a flow control mechanism that automatically and selectively increases flowable communication between this substance and the person's oral cavity or nasal cavity when a selected nutrient or food is being digested within the person's mouth. This invention can help a person to moderate their consumption of unhealthy food and to limit their overall caloric intake as part of a comprehensive system for improved nutrition, energy balance, and weight management.

Automatic and selective modification of the taste and/or smell of selected nutrients or foods in order to reduce consumption of unhealthy food, as disclosed herein, does not appear to be disclosed or anticipated by the prior art. Further, such automatic and selective modification of the taste and/or smell of unhealthy food, as food is being digested in the mouth, offers several advantages over the prior art.

As one advantage, unlike prior art that relies on consistent voluntary compliance by the person in order to work, the implantable invention that is disclosed herein operates automatically to modify food consumption. This invention does not depend on consistent voluntary compliance by the person. As another advantage, unlike prior art that is blind to whether food is healthy or unhealthy, the implantable invention that is disclosed herein can differentiate between healthy vs unhealthy food. This invention can selectively modify consumption of unhealthy food. This invention does not run the risk of depriving the person of essential nutrients like prior art that blindly reduces consumption and/or absorption of healthy food as well as unhealthy food.

### INTRODUCTION TO THE FIGURES

FIGS. 1 through 23 show several examples of how this invention can be embodied to automatically and selectively modify the taste and/or smell of one or more selected nutrients or foods, as a person digests food in their mouth. These figures do not limit the full generalizability of the claims.

FIG. 1 shows an example of a device that modifies the taste and/or smell of a selected nutrient or food within a person's mouth wherein this device is attached to, or implanted within, the person's palatal vault.

FIG. 2 shows how such a device can communicate wirelessly with an external control unit.

FIG. 3 shows an example of a device that modifies the taste and/or smell of a selected nutrient or food within a person's mouth wherein this device is attached to, or implanted within, the person's tongue.

FIG. 4 shows an example of a device that modifies the taste and/or smell of a selected nutrient or food within a person's mouth wherein part of this device is implanted within the person's chest.

FIGS. 5 through 7 provide a three-figure sequence showing how such a device and method can encourage a person to eat less of a selected type of food.

FIGS. 8 through 11 provide a four-figure sequence showing how a taste and/or smell modifying device with multiple compartments can selectively encourage a person to eat less of a selected type of food.

FIGS. 12 through 14 show an example of a device that modifies the smell of a selected nutrient or food wherein this device is attached to, or implanted within, the person's nasal cavity.

FIG. 15 shows an example of how a peristaltic pump can be used as part of this invention.

FIG. 16 shows an example of how this invention can be embodied in a three-step method to modify the smell and/or taste of a selected nutrient or food while food is being digested in a person's mouth.

FIG. 17 shows an example of how this invention can be embodied in a four-step method to modify the smell and/or taste of a selected nutrient or food after more than a moderate amount of such nutrient or food has been consumed.

FIGS. 18 through 21 show, in greater detail, how a taste and/or smell modifying device can automatically and selectively discourage consumption of unhealthy food without discouraging consumption of healthy food.

FIG. 22 shows an example of how this invention can be embodied in a two-step method for modifying the taste and/or smell of one or more selected nutrient or food types while food is being digested in a person's mouth.

FIG. 23 shows an example of how this invention can be embodied in a two-step method for modifying the taste and/or smell of an unhealthy type or quantity of food, as food is being digested in a person's mouth.

### DETAILED DESCRIPTION OF THE FIGURES

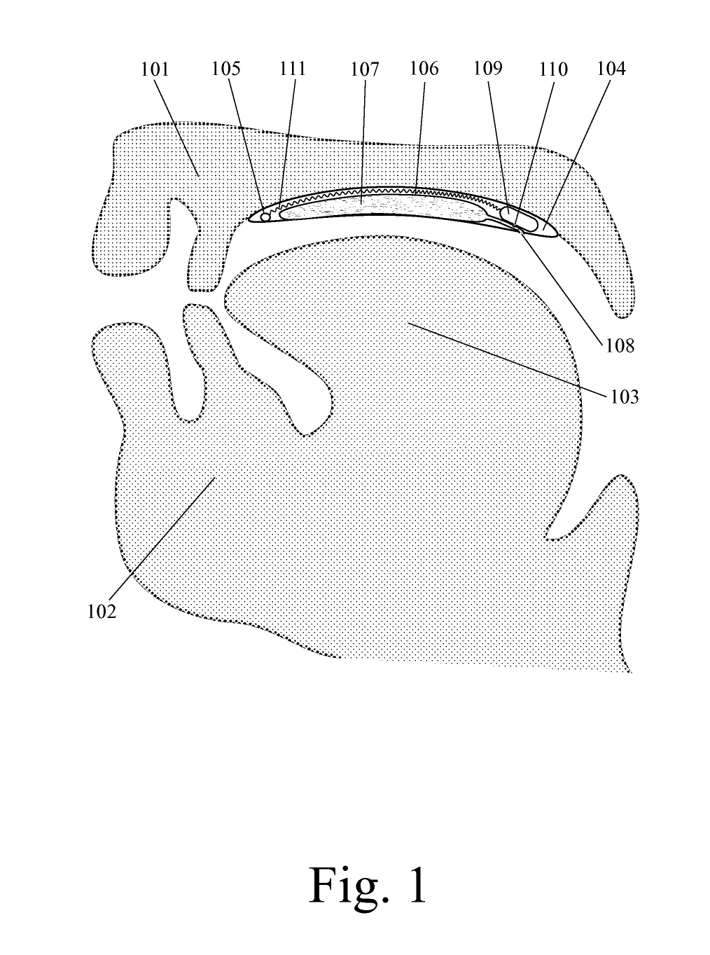

FIGS. 1 through 23 show some examples of how this invention can be embodied in a device and method to automatically and selectively modify the taste and/or smell of one or more selected nutrients or foods, as a person digests food in their mouth, in order to change the person's eating patterns. This, in turn, can improve the person's nutrition and help them to manage their weight. However, these figures are just some examples of how this invention can be embodied. They do not limit the full generalizability of the invention claims.

FIG. 1 shows an example of how this invention can be embodied in an implantable device that modifies the taste and/or smell of a selected nutrient or food as that nutrient or food is being digested within a person's mouth. This real-time modification of a food's taste and/or smell, while it is being consumed, can change the person's eating habits so that they have improved nutrition and can better manage their weight. In the example shown in FIG. 1, a device is attached to the palatal vault on the upper roof of the person's mouth. From this location, this device can detect one or more selected nutrients or foods, based on analysis of saliva, in order to modify the taste and/or smell of those nutrients or foods as they are being digested within the person's mouth.

FIG. 1 shows this device in a lateral cross-sectional view of the person's mouth. This view includes the upper portion 101 of the person's oral cavity (including the upper jaw, upper teeth, upper gums, and palatal vault), the lower portion 102 of the person's oral cavity (including the lower jaw, lower teeth, and lower gums), and the person's tongue 103. In this example, an automatic taste-modifying device is attached to, or implanted within, the palatal vault of the upper portion 101 of the person's oral cavity.

In the example shown in FIG. 1, an automatic taste-modification device comprises: a housing 104 which is attached to the palatal vault of the upper portion 101 of the person's oral cavity; a nutrient or food sensor 105 in housing 104 which is in flowable communication with saliva in the person's oral cavity; a liquid taste and/or smell modifying substance 107; a substance reservoir 106 which contains the taste and/or smell modifying substance 107 until it is released; an opening 108 between reservoir 106 and the person's oral cavity through which the taste and/or smell modifying substance 107 can flow; a flow control mechanism 109 which automatically and selectively controls the flow of substance 107 through opening 108; a valve 110 which is controlled by flow control mechanism 109; and a wire 111 which transmits signals from sensor 105 to flow control mechanism 109.

Before discussing the individual components of the embodiment in FIG. 1 in detail, we first present an overview of the sequence of operations for this embodiment. First, when a person digests a selected nutrient or food type within their mouth, the nutrient or food sensor 105 automatically and selectively detects digestion of this nutrient or food type. Second, signals indicating digestion of this selected nutrient or food type are conveyed from the sensor 105 to the flow control mechanism 109 through wire 111.

Third, depending on its programming, flow control mechanism 109 can open valve 110 in order to allow the taste and/or smell modifying substance 107 to come into fluid and/or gaseous communication with the person's oral cavity and/or nasal cavity.

Next, the fluid and/or gaseous communication between the taste and/or smell modifying substance 107 and the person's oral and/or nasal cavity modifies the taste and/or smell of the food as it is being digested in the person's mouth. This can change the person's eating habits with respect to this nutrient or food type. Decreased consumption of deleterious nutrients or foods and/or increased consumption of beneficial nutrients or foods can help the person to improve their nutrition and better manage their weight.

We now discuss some of the components of the embodiment shown in FIG. 1 in greater detail. This discussion includes several alternative examples and variations for these components which are within the scope of this invention's claims. The components discussed herein include: the nutrient or food sensor; the taste and/or smell modifying substance; the substance reservoir; and the flow control mechanism.

We start by discussing the nutrient or food sensor in greater detail. In the example of this invention that is shown in FIG. 1, the nutrient or food sensor 105 is in fluid communication with saliva in the person's oral cavity. This sensor 105 detects digestion of one or more selected nutrients or food types in the person's mouth. In this example, nutrient or food sensor 105 detects a selected nutrient or food type by analyzing the composition of the person's saliva. In this example, nutrient or food sensor 105 is a chemical sensor that uses chemical analysis to identify particular nutrients and/or food types. In this example, nutrient or food sensor 105 analyzes the composition of the person's saliva to automatically and selectively detect when a person is digesting a food that is high in sugar while that food is being digested within the person's mouth.

In various examples, a nutrient or food sensor can automatically and selectively detect high amounts or concentrations of one or more nutrients selected from the group consisting of: sugar or simple carbohydrates in general, saturated fat or fats in general, cholesterol, salt, protein, and fiber. In various examples, a sensor can automatically and selectively detect digestion of certain types of food with high amounts of, or high proportions of calories in the form of: sugar or simple carbohydrates in general, saturated fat or fats in general, cholesterol, salt, protein, and/or fiber.

In an example, one or more selected nutrients or food types, whose taste and/or smell is modified by this invention, can be selected from the group consisting of: fried or deep-fried food, French fries, cholesterol or high-cholesterol food, fat or high-fat food or high-saturated-fat food, high-fructose corn syrup, salt or high-sodium food, simple or refined sugar or high-sugar food, hydrogenated oil, and soda pop. In an example, unhealthy nutrients and food may be identified by being in the group consisting of: fried or deep-fried food, French fries, cholesterol or high-cholesterol food, fat or high-fat food or high-saturated-fat food, high-fructose corn syrup, salt or high-sodium food, simple or refined sugar or high-sugar food, hydrogenated oil, and soda pop.

In various examples, a nutrient or food sensor can be selected from the group consisting of: a chemical or biochemical sensor, an enzymatic sensor, a biological sensor, a cholesterol sensor, a fat sensor, a glucose sensor, an impedance sensor, a interferometer sensor, a membrane sensor, a Micro Electrical Mechanical System (MEMS) sensor, a microfluidic sensor, a nanoparticle sensor, a nanoscale sensor, a neural sensor, an electromagnetic sensor, a micronutrient sensor, an optical sensor, a protein sensor, a reagent sensor, a genetic sensor, a sonic or ultrasonic sensor, and a strain sensor.

In various examples, the mechanism by which a nutrient or food sensor identifies one or more specific nutrients or food types can be selected from the group consisting of: biochemical-based sensor, biological sensor, chemical-based sensor, cholesterol-based sensor, enzyme-based sensor, fat sensor, filtration-based sensor, genetic sensor, glucose sensor, interferometer-based sensor, membrane-based sensor, Micro Electrical Mechanical System (MEMS) sensor, microfluidic sensor, nanoparticle-based sensor, nanoscale sensor, neural sensor, electromagnetic sensor, micronutrient sensor, optics-based sensor, chromatography-based sensor, protein-based sensor, reagent-based sensor, and strain sensor. In various examples, a sensor can be a “laboratory on a chip” or a “medichip.”

In the example shown in FIG. 1, nutrient or food sensor 105 is partially contained within housing 104 and is also in fluid communication with the person's oral cavity. Housing 104, in turn, is configured to be attached to, or implanted within, the person's body. In this example, housing 104 is attached to the person's palatal vault on the roof of their mouth. In this example, housing 104 is attached with a removable dental adhesive. In an example, housing 104 can be removably attached with a clip or snap to a component that is permanently attached to the underlying hard palate. In this example, the tip of sensor 105 is on the surface of housing 104 in order to be in fluid communication with saliva in the person's oral cavity.

In various examples of this device, a nutrient or food sensor, or the housing for such a sensor, can be configured to be attached to, or implanted within, other tissue structures that comprise or surround the person's oral cavity. For example, a sensor, or the housing for a sensor, can be configured to be attached to, or implanted within, the person's teeth. In various examples, a sensor can be attached to the lingual, palatal, buccal, and/or labial surfaces of a person's teeth. In an example, a nutrient or food sensor can be incorporated into a dental and/or orthodontic appliance. In an example, a nutrient or food sensor can be incorporated into a dental bridge, cap, or crown.

In another example, a sensor, or the housing for a sensor, can be configured to be attached to, or implanted within, or attached underneath the person's tongue. In an example, a nutrient or food sensor could be inserted into a person's tongue. In an example, a sensor could be attached or implanted sublingually. In another example, a sensor can be configured to be attached to, or inserted into, the soft palate tissues at the rear of the person's oral cavity.

In another example, a nutrient or food sensor can be in fluid and/or gaseous communication with the person's nasal cavity. In an example, a nutrient or food sensor can detect a selected nutrient or food type as it is being digested in the person's mouth based on gaseous emissions (e.g. smells) from the food that flow into the person's nasal cavity. In an example, a nutrient or food sensor can be in gaseous communication with the person's nose or sinuses.

We now discuss the taste and/or smell modifying substance in greater detail. The example of this invention that is shown in FIG. 1 includes a taste and/or smell modifying substance 107 that modifies, masks, and/or blocks the taste and/or smell of food as food is digested within the person's mouth. This taste and/or smell modification only occurs when the substance is brought into fluid and/or gaseous communication with the person's oral cavity and/or nasal cavity. This allows taste or smell modification to be targeted to a particular nutrient or food at the point of initial digestion. This, in turn, allows considerable precision in modifying a person's eating habits in order to improve their nutrition and help them manage their weight.

In an example, a taste and/or smell modifying substance can interact with the person's taste buds and/or olfactory receptors. In an example, this interaction can occur through direct administration of the substance to the person's taste buds and/or olfactory receptors. In an example, this interaction can occur through diffusion of the substance through the person's saliva or inhaled air. Since this substance is only exposed to the person's oral cavity and/or nasal cavity when a person digests a particular nutrient or food type, this selective exposure comprises immediate and targeted dietary feedback. Immediate and targeted dietary feedback of this type can directly modify the person's consumption of a particular nutrient or food type.

In an example, a taste and/or smell modifying substance 107 can have a very strong taste and/or smell that overpowers the normal taste and/or smell of one or more selected foods. In the example shown in FIG. 1, the taste and/smell modifying substance can have a strong bitter taste. In various examples, a taste and/or smell modifying substance can have a taste and/or smell that is very bitter, sour, spicy, hot . . . or just plain noxious. This can actively discourage consumption of food, at the time of initial ingestion, as the taste and/or smell modifying substance comes into fluid and/or gaseous communication with the person's oral cavity and/or nasal cavity.

In an example, the taste and/or smell of the taste and/or smell modifying substance need not be bad in itself in order to accomplish this purpose. The taste and/or smell might just interfere with the normally-expected taste and/or smell of a particular food in a way that makes that particular food unappetizing. For example, peppermint oil is a strong flavor that need not be bad. Many people appreciate peppermint flavor in moderation, particularly for candies or ice cream. However, a strong peppermint oil would be a peculiar, and probably undesirable, additive during the digestion of French fries in a person's mouth. The release of peppermint oil by a device during the digestion of French fries in a person's mouth would probably cause the person to get a funny look on their face (“Peppermint-flavored French fries? Gross!”) and eat fewer French fries.

In a like manner, many people appreciate the taste of cloves when eating ham, but would not enjoy the release of clove oil into their mouth while eating ice cream (“Clove-flavored ice cream? Gross!”). Finally, as an olfactory example, many people enjoy “new car smell” in its proper context—a car. However, people would probably not enjoy “new car smell” when eating pizza. The gustatory and olfactory contexts of tastes and smells can be important elements of how they are perceived and their behavioral impact on diet. Tangentially, there is also the possibility that this device could create a new taste fad (peppermint-flavored French fries or clove-flavored ice cream!) in which case more aversive flavors would have to be found.

In an example, a bad tasting and/or bad smelling substance can be selected so that a person eats less unhealthy food when this substance comes into fluid and/or gaseous communication with their oral cavity and/or nasal cavity. The immediate and targeted release of an unpleasant taste or smell in response to the consumption of a particular food type can powerfully change a person's eating habits for the better. A device or method that modifies the taste and smell of food in this manner can provide strong support for a person's willpower during challenging times and moments of dietary weakness. For example, a person's willpower might be low in the middle of the night. They might be tempted to binge on ice cream. However, the release of cod liver oil in response to ice cream consumption at night can rapidly dampen such midnight craving.

The ability of such a device or method to target specific foods can be an improvement over prior art that blindly limits the volume of all food consumed and cannot differentiate between different types of (e.g. less-healthy vs. more-healthy) food. The device and method disclosed herein is a significant improvement over prior art that blindly constricts the consumption volume, consumption rate, or digestive absorption of all food consumed, regardless of whether the food is healthy or unhealthy.

In an example, a device and method for dietary modification like the one disclosed herein can be programmed to allow moderate consumption of some nutrients or foods, but to discourage excessive consumption of those nutrients or foods. Some nutrients or food types can be fine in moderation, but harmful in excess. For example, a device might only release a taste and/or smell modifying substance if the person consumes more than a certain amount of a selected nutrient or food type during a selected period of time. This can allow a person to consume a moderate amount of the selected nutrient or food type without any taste and/or smell modification, but still limit over-consumption or binge eating. Dietary moderation, as opposed to complete prohibition of certain foods, can make a dietary regimen (and thus the related device or method) more acceptable to a user.

In another example, a device and method for dietary modification such as the one disclosed herein can be programmed to only modify the taste and/or smell of selected nutrients or foods at certain times of the day. In an example, the device may only a release a taste-modifying substance in response to the person's eating selected nutrients or foods when such eating occurs between regularly-scheduled meals. This can discourage between-meal snacking. In an example, a device can release a taste-modifying substance in response to the person's eating selected nutrients or foods only at night. This can discourage nocturnal eating binges.

In another example, a device and method for dietary modification such as the one disclosed herein can be programmed to allow unmodified consumption of selected nutrients or foods, for a limited time period or up to a certain amount, as a reward when the person meets his or her exercise and/or weight loss goals. In another example, a device such as the one disclosed herein can be programmed to temporarily allow unmodified consumption of selected nutrients or foods during special social eating events and/or holiday meals.

In another example, a device and method for dietary modification such as the one disclosed herein can be programmed to only change the taste and/or smell modification of food at certain geographic locations. In an example, the device may communicate with a GPS system and discourage eating in certain locations which are associated with unhealthy consumption behaviors.

In another example, a device and method for dietary modification such as the one disclosed herein can be programmed to link taste and/or smell modification to financial incentives and penalties. In an example, a person may receive a discount off their health insurance premium as a reward for successful dietary modification. In another example, a device and method for dietary modification such as the one disclosed herein can be programmed to communicate with a social support network to provide a person with support for their willpower during challenging times or moments of dietary weakness. In addition to discouraging or limiting consumption of unhealthy foods, the device and method disclosed herein can also be used to encourage greater consumption of healthy foods. For example, a good tasting and/or good smelling substance can be selected so that a person eats more food when the substance comes into fluid and/or gaseous communication with their oral cavity and/or nasal cavity. In various examples, a good tasting and/or smelling substance can have a taste and/or smell that is appetizing, pleasant, sweet, or savory. This can encourage a person's consumption of beneficial nutrients and food types.

In an example, if there are certain foods that would greatly improve a person's health, but which the person normally finds unappealing, then this device can improve, mask, or block the perceived taste and/or smell of those foods in order to encourage the person to eat more of them. For example, if the person should eat more dark-green vegetables, but finds them unappetizing, then the device could release a substance that improves, masks, or blocks the flavor of such food as it is consumed. In the extreme, if a person really dislikes the flavor of a particularly-healthy food, then the device could release a substance that temporarily limits the operation of taste buds and/or olfactory receptors as the healthy food is consumed. In an example, a device could keep track of consumption of particular types of healthy food, via the nutrient or food sensor, and could reward the consumption of good food with limited amounts of allowable consumption of bad food (without taste modification). Perhaps some people are willing to eat more broccoli for some chocolate?

In various examples, a taste and/or smell modifying substance can be stored and released in various formulations. In the example of the invention that is shown in FIG. 1, the taste and/or smell modifying substance 107 is stored within reservoir 106 in liquid form. It also remains in liquid form as it is released into the person's oral cavity to blend with the person's saliva and reach the person's taste buds. In various examples, a taste and/or smell modifying substance can be stored as a liquid, gel, foam, powder, granules, solid, or gas. In various examples, a taste and/or smell modifying substance can be released into the person's oral cavity and/or nasal cavity as a liquid, gel, foam, powder, granules, solid, or gas. In an example, a taste and/or smell modifying substance can be contained within small capsules which only dissolve in the person's mouth when they are exposed to a selected nutrient or food type.

In an example, a taste and/or smell modifying substance can be Generally Recognized As Safe (GRAS) under Sections 201(s) and 409 of the Federal Food, Drug, and Cosmetic Act. In any event, the substance should be safe and non-toxic. In an example, a taste and/or smell modifying substance can be a naturally-occurring substance. In an example, a taste and/or smell modifying substance can be a plant oil or a plant extract. In an example, a taste and/or smell modifying substance can be a high-concentration formulation of a food additive that is commonly used to make food taste bitter, sour, sweet, salty, or savory. In an example, a taste and/or smell modifying substance can be a spice. In an example, a taste and/or smell modifying substance can be a substance for which people give a high score on an empirically-validated scale of taste intensity.

In an example, a taste and/or smell modifying substance can be selected to cause temporary ageusia and/or anosmia. In an example, a taste and/or smell modifying substance can temporarily anesthetize taste buds and/or olfactory receptors. In an example, the substance can have a localized, non-toxic, anesthetic effect. In an example, a taste and/or smell modifying substance can coat taste buds and/or olfactory receptors to temporarily reduce contact between them and food during digestion. In an example, a taste and/or smell modifying substance can temporarily modify nerve impulses traveling from taste buds and/or olfactory receptors to the brain, in order to modify the perception of taste and/or smell. In an example, a taste and/or smell modifying substance can create the perception of a strong taste and/or smell by neural stimulation, without coming into direct flowable communication with the person's taste buds and/or olfactory receptors. In various specific examples, a taste and/or smell modifying substance can be selected from the group consisting of: Acetic Acid, Almond Oil, Amaranth Oil, Anise Flavor, Apple Seed Oil, Apricot Oil, Argan Oil, Artichoke Flavor, Asparagus Flavor, Avocado Oil, Babassu Oil, Barley Flavor, Basil Flavor, Beech Nut Oil, Bitter Gourd Oil, Black Pepper Oil, Black Seed Oil, Blackcurrant Seed Oil, Bleu Cheese Flavor, Borage Seed Oil, Bottle Gourd Oil, Brine, Broccoli Flavor, Buffalo Gourd Oil, Camellia Oil, Canola Oil, Cape Chestnut Oil, Cardamom Flavor, Carob Oil, Cashew Oil, Castor Oil, Cayenne Powder, Celery Seed Flavor, Cheese Cake Flavor, Cherry Flavor, Chili Powder, Chocolate Flavor, Cilantro Flavor, Cinnamon Oil, Citric Acid, Citrus Oils, Clove Oil, Clove Powder, Cocklebur Oil, Cocoa Flavor, Coconut Oil, Cod Liver Oil, Coffee Flavor, Cohune Oil, Colza Oil, Coriander Seed Oil, Corn Oil, Cottonseed Oil, Cumin Powder, Date Seed Oil, Dextrin, Dextrose, Dika Oil, Eucalyptus Oil, False Flax Oil, Fennel Oil, Fennel Powder, Fish Oil, Flaxseed Oil, Fructose, Fumaric Acid, Galactose, Garlic Oil, Ginger Powder, and Ginger Oil.

In various specific examples, a taste and/or smell modifying substance can be selected from the group consisting of: Glucose, Gorgonzola Cheese Flavor, Grape Flavor, Grape Seed Oil, Grapefruit Flavor, Grapefruit Seed Oil, Hazelnut Oil, Hemp Oil, Horseradish Flavor, Ipecac, Isocyanide, Isonitrile, Jasmine Flavor, Kapok Seed Oil, Kenaf Seed Oil, Lactose, Lallemantia Oil, Lavender Flavor, Lecithin, Lemon Juice, Lemon Myrtle Flavor, Lemon Oil, Licorice Flavor, Lime Juice, Lime Oil, Linseed Oil, Lutefisk Flavor You Betcha, Macadamia Oil, Mafura Oil, Malic Acid, Maltodextrin, Maltose, Mango Flavor, Marula Oil, Menthol Oil, Mint Oil, Mongongo Nut Oil, Mustard Flavor, Mustard Oil, Nutmeg Oil, Okra Seed Oil, Olive Oil, Onion Flavor, Orange Flavor, Orange Oil, Oregano Flavor, Palm Oil, Papaya Seed Oil, Peanut Oil, and Pecan Oil.

In various specific examples, a taste and/or smell modifying substance can be selected from the group consisting of: Pepper Oil, Peppermint Oil, Pequi Oil, Perilla Seed Oil, Persimmon Seed Oil, Pili Nut Oil, Pimento Flavor, Pine Nut Oil, Pistachio Oil, Pomegranate Seed Oil, Poppyseed Oil, Prune Kernel Oil, Pumpkin Seed Oil, Quinine Sulfate, Quinoa Oil, Radish Oil, Ramtil Oil, Rapeseed Oil, Raspberry Flavor, Rice Bran Oil, Rosemary Flavor, Royle Oil, Saccharin, Safflower Oil, Sage Flavor, Salicornia Oil, Saline Solution, Sapote Oil, Seje Oil, Sesame Oil, Shea Butter, Sodium Chloride, Sorbitol, Soybean Oil, Spearmint Oil, Strawberry Flavor, Succinic Acid, Sucrose, Sunflower Oil, Tamarind Flavor, Taramira Oil, Tartaric Acid, Thistle Oil, Tigernut Oil, Tobacco Seed Oil, Tomato Seed Oil, Vanilla Flavor Acai Oil, Walnut Oil, Watermelon Seed Oil, Wheat Germ Oil, Yangu Oil, and Yeast Extract.

Now we discuss the substance reservoir in more detail. The example of this invention that is shown in FIG. 1 includes substance reservoir 106 in which taste modifying substance 107 is stored until it is brought into fluid communication with the person's oral cavity. In this example, substance reservoir 106 is contained within housing 104 and housing 104 is configured to be attached to, or implanted within, the person's body. In this example, the housing 104 for substance reservoir 107 is configured to be attached to, or implanted within, the tissue comprising the person's oral cavity.

In this example, reservoir 106 and/or housing 104 are relatively thin and flexible. In this example, housing 104 has a lubricious and biocompatible coating so that it does not irritate the person's tongue or otherwise bother the person within their mouth. In an example, housing 104 for reservoir 106 can be attached to the surface of the person's palatal vault. In an example, housing 104 can be implanted under the surface tissue of the person's palatal vault, with sensor 105 and opening 108 on the surface for fluid communication with the person's oral cavity.

In an example, housing 104 and reservoir 107 can be flexible and elastic. In an example, pressure from the elasticity of reservoir 107 can cause substance 107 to leak out when valve 110 is opened. In an example, pressure on the flexible housing 104 and reservoir 106 from the person's tongue (and from food during consumption) compress the housing 104 and reservoir 106, thereby causing substance 107 to leak out of reservoir 106 when valve 110 is opened. In another example, reservoir 106 and housing 104 can be rigid and uncompressible. In an example, substance 107 can be released into the oral cavity by active pumping or spraying means, wherein this active pumping or spraying is controlled by flow control mechanism 109.

In another example, the substance reservoir and/or its housing can be configured to be attached to, or implanted within, the person's nasal cavity or tissue surrounding the person's nasal cavity. In another example, the substance reservoir and/or its housing can be configured to be attached to, or implanted within, the person's chest, abdominal cavity, and/or torso. If the reservoir and/or its housing is not directly in fluid and/or gaseous communication with the person's oral cavity and/or nasal cavity, then the reservoir can be connected to the person's oral cavity and/or nasal cavity by means of a tube or other lumen. In this manner, a taste and/or smell influencing substance can still flow from the reservoir into the person's oral cavity and/or nasal cavity.

In various examples, a reservoir that contains a taste and/or smell modifying substance, or the housing for that reservoir, can be configured to be: attached to the person's oral cavity or tissue surrounding the oral cavity; implanted within the person's oral cavity or tissue surrounding the oral cavity; attached to the person's nasal cavity or tissue surrounding the nasal cavity; implanted within the person's nasal cavity or tissue surrounding the nasal cavity; or implanted within the person's chest, abdominal cavity, and/or torso and in fluid and/or gaseous communication with the person's oral cavity and/or nasal cavity via a tube or other lumen.

In an example, a substance reservoir (and its housing) can be attached to, or implanted within, a person's body. In an example, a reservoir and/or its housing can be attached to the tissue surface that comprises the person's oral cavity or nasal cavity. In the example shown in FIG. 1, the housing for substance reservoir 106 is attached to the palatal vault of the upper portion of the person's oral cavity. In this example, the housing is attached to palatal vault tissue with a removable dental adhesive. In an example, the housing could be attached to the palatal vault by covering it with adhesive tape, an adhesive patch, or a biocompatible mesh.

In another example, a reservoir and/or its housing can be inserted or imbedded into the tissue of the palatal vault tissue. In an example, the reservoir and/or its housing can be attached to the underlying hard palate using an adhesive, a rotating fastener, or sutures. In various examples, a substance reservoir, or the housing for such a reservoir, can be configured to be attached to, or implanted within, the person's body by a means selected from the group consisting of: adhesive and/or glue; adhesive tape or patch; clamp, clip, and/or snap; elastic band or other elastic member; frictional engagement with tissue; hook and eye materials with attachable layers; sutures, staples, and pins; and screws or other rotating fasteners. In various examples, a reservoir and/or its housing can be comprised of a member selected from the group consisting of: a palatal vault implant; a strip, patch, or pad; a dental appliance; a mouth guard; and a tongue implant.

In an example, there can be a sensor or gauge within a reservoir that detects and communicates how much taste and/or smell modifying substance is contained within the reservoir. In an example, a substance reservoir can be refilled with a taste and/or smell modifying substance by complete removal and replacement of the reservoir and/or its housing. In an example, a reservoir and/or housing can be disposable rather than refillable. In an example, a substance reservoir can be refilled with substance by direct injection of the substance into the reservoir, such as through an elastic or rubber membrane. In an example, a substance reservoir can be refilled by removal and replacement of a substance-containing cartridge that is inserted into the reservoir.

In this example, substance reservoir 106 has only one compartment that contains only one type of taste and/or smell modifying substance. In another example, a substance reservoir can have multiple compartments that contain separate doses of the same taste and/or smell modifying substance. In another example, a substance reservoir can have multiple compartments that contain different taste and/or smell modifying substances. In an example, the device and method disclosed herein can release different types or quantities of taste and/or smell modifying substances in response to digestion of different types of nutrients or foods.

In an example, a substance reservoir can have multiple compartments that are selectively brought into fluid and/or gaseous communication with a person's oral cavity and/or nasal cavity in response to consumption of different types of nutrients or foods. In an example, a substance reservoir can have multiple compartments that are sequentially brought into fluid and/or gaseous communication with a person's oral cavity and/or nasal cavity. In an example, the device can be programmed to determine which types of substances are released within the mouth in response to which types of food, when they are released, and how they are released.

In an example, a substance reservoir can have multiple compartments that are brought into fluid and/or gaseous communication with a person's oral cavity and/or nasal cavity at different times. In an example, the substance reservoir can have different compartments that are brought into fluid and/or gaseous communication with a person's oral cavity and/or nasal cavity in order to provide discrete doses of a taste and/or smell modifying substance when the person digests a selected nutrient or food type within their mouth consumption at different times.

In an example, a device may release a similar amount of a taste and/or smell modifying substance into the person's oral cavity each time that the person consumes a given type of food. In an example, this device and method may be designed to release the same dose of a taste and/or smell modifying substance each time that the person consumes a particular type of food. In an example, equal dosing may be achieved by having separate compartments of roughly equal size be sequentially exposed to the person's oral cavity. In an example, equal dosing may be achieved through use of a peristaltic pump, electromagnetic pump, or MEMS pump that is controlled by the flow control mechanism.

In an example, a device may release increasing amounts of a taste and/or smell modifying substance into the person's oral cavity as the person eats more and more of a given type of food during a period of time. In an example, a device may release different types of taste and/or smell modifying substances into the person's oral cavity as the person eats more and more of a given type of food during a period of time. In an example, complex patterns of automatic release of different substances in response to consumption of different types of foods can be adjustably programmed into such a device.

In an example, different compartments within a reservoir can hold a limited number of different “primary” or “elemental” taste or smell substances. These “primary” or “elemental” taste or smell substances can be blended together by the device to create a wide spectrum of complex tastes and smells. Such blending is analogous to the way in a limited number of “primary” colors can be blended together to create the whole spectrum of colors. The combined effect of two or more “primary” or “elemental” taste or smell substances can be greater than the sum of their individual effects. In an example, each compartment of the reservoir may have its own valve and/or pump to allow customized, real-time, blending of “primary” or “elemental” taste or smell substances to create a whole spectrum of complex tastes and smells.

In an example, there can be multiple openings between a reservoir, or compartments within that reservoir, and the person's oral cavity. In various examples, releasing one or more substances from openings in different areas of the person's oral cavity may have different taste effects as food is digested. In an example, releasing one or more substances into the mouth through multiple openings may have a greater effect than releasing a substance through only one opening. In an example, a device can simultaneously release a taste-modifying substance into the person's mouth and a smell-modifying substance into the person's nose. These two actions together can combine to cause greater behavior-changing effect than the sum of the individual effects of such actions.

We now discuss the flow control mechanism in greater detail. In an example, a flow control mechanism, such as 109 in FIG. 1, can change the degree of fluid and/or gaseous communication between a taste and/or smell modifying substance and a person's oral cavity and/or nasal cavity, based on the person's digestion of a selected nutrient or food type in their mouth, in order to change the person's consumption of that nutrient or food type. In an example, a flow control mechanism can be part of an overall system to help a person improve their nutrition, maintain energy balance, and manage their weight.

In some respects, the flow control mechanism is the “brains” of this device. In some examples, a flow control device can be programmed to modify the taste and/or smell of selected foods to improve a person's eating habits. Such improvements can include reduced or limited consumption of unhealthy foods, avoidance of binges and overeating, and increased consumption of healthy foods.

Although there are some important differences between this present device (that modifies diet) and a pacemaker (that modifies heart rhythm), they both monitor and enhance important body functions. This is why we use the term “tastemaker” (TM) to describe the invention disclosed herein. Also, it is a catchy name. While pacemakers (for one's heart) have been in use for several decades, the “tastemaker” (for one's diet) disclosed herein is a novel approach for addressing the growing problems of poor nutrition and obesity.

There are some interesting similarities and some important differences between a pacemaker (for one's heart) and a “tastemaker” (for one's diet). A pacemaker (for one's heart) uses electrical impulses to help a person's heart beat at the proper rhythm, neither too fast nor too slow. The “tastemaker” (for one's diet) disclosed herein uses taste and/or smell modifying substances to help a person consume foods in healthy proportions, neither too much nor too little. A pacemaker (for one's heart) can be programmed to adjust the heart's rhythm based on a person's exercise patterns and based on diagnostic evaluation by a clinician. In an example, the “tastemaker” (for one's diet) disclosed herein can be adjusted based on the person's exercise patterns and based on diagnostic evaluation by a clinician. A pacemaker (for one's heart) can be wirelessly programmed. In an example, the “tastemaker” (for one's diet) disclosed herein can be wirelessly programmed.

During the past couple decades, there has been considerable progress in the evolution of implantable pacemakers (and automatic defibrillators) to monitor and manage the beating heart. Perhaps during the next decade there will be considerable progress in the evolution of implantable tastemakers to monitor and manage personal diet and nutrition? The implantable device and method that is disclosed herein is a start.

In the example of this invention shown in FIG. 1, a taste and/or smell modifying substance 107 is brought into fluid communication with the person's oral cavity when the flow control mechanism 109 opens valve 110. This allows the liquid substance 107 to flow from reservoir 106 through opening 108 into the person's oral cavity and/or nasal cavity where it comes into contact with the person's taste buds.

In an example, a taste and/or smell modifying substance can be brought into fluid communication with the person's oral cavity by passive osmosis of the substance from an implanted reservoir into the person's oral cavity. In an example, a taste and/or smell modifying substance can be brought into fluid communication with the person's oral cavity by passive osmosis of saliva from the person's oral cavity into an implanted reservoir. In other examples, a taste and/or smell modifying substance can be brought into fluid communication with the person's oral cavity by active pumping or spraying of the substance from an implanted reservoir into the person's oral cavity. In an example, a flow control mechanism can control the degree of fluid and/or gaseous communication between a taste and/or smell modifying substance and a person's oral cavity and/or nasal cavity by accurately pumping or spraying a selected amount of the substance from an implanted reservoir into the person's oral cavity and/or nasal cavity.

In the example of this invention that is shown in FIG. 1, flow control mechanism 109 automatically and selectively increases the fluid and/or gaseous communication between the taste and/or smell modifying substance 107 and the person's oral cavity when the nutrient or food sensor 105 detects that the person is digesting a selected nutrient or food type within their mouth. In an example, a flow control mechanism controls the degree of fluid and/or gaseous communication between a taste and/or smell modifying substance and a person's oral cavity and/or nasal cavity by opening (or closing) a valve between an implanted reservoir that contains the substance and the person's oral cavity and/or nasal cavity.

In various examples, a flow control mechanism can control the degree of fluid and/or gaseous communication between a taste and/or smell modifying substance and a person's oral cavity and/or nasal cavity by opening or closing a valve, opening, plug, flap, or lumen between an implanted reservoir (that contains the substance) and the person's oral cavity and/or nasal cavity. In various examples, a flow control mechanism can control the degree of fluid and/or gaseous communication between a taste and/or smell modifying substance and a person's oral cavity and/or nasal cavity by actively pumping or spraying the substance from an implanted reservoir into the person's oral cavity and/or nasal cavity.

In various examples, a flow control mechanism can change the amount, timing, duration, or location of fluid and/or gaseous communication between a taste and/or smell modifying substance and the person's oral cavity and/or nasal cavity. In an example, a flow control mechanism can be programmed to change the degree to which a taste and/or smell modifying substance is brought into fluid and/or gaseous communication with the person's oral cavity and/or nasal cavity based on the person's past eating patterns.

In an example, a flow control mechanism can be programmed to change the degree to which a taste and/or smell modifying substance is brought into fluid and/or gaseous communication with the person's oral cavity and/or nasal cavity based on the person's past exercise patterns. In an example, the flow control mechanism can be part of an overall system of behavioral modification for proper nutrition, energy balance, and weight management.

In an example, a flow control mechanism can be programmed to change the manner in which a taste and/or smell modifying substance is brought into fluid and/or gaseous communication with the person's oral cavity and/or nasal cavity based on the time of day. This can discourage snacking between meals or binge eating at night.

In an example, a flow control mechanism can be programmed to change the manner in which a taste and/or smell modifying substance is brought into fluid and/or gaseous communication with the person's oral cavity and/or nasal cavity based on the person's success in meeting weight or exercise targets. This can reward successful weight management.

In an example, a flow control mechanism can be programmed to change the manner in which a taste and/or smell modifying substance is brought into fluid and/or gaseous communication with the person's oral cavity and/or nasal cavity based on wireless communication with a diet coach or other health care professional, caretaker, friend or social support network, and/or external computing device or the Internet. This can provide a personal support network to strengthen a person's willpower during challenging times or moments of dietary weakness.

In an example, a flow control mechanism can automatically increase the fluid and/or gaseous communication between a taste and/or smell modifying substance and a person's oral cavity and/or nasal cavity when the person digests a selected nutrient or food type within their mouth. In an example, a flow control mechanism can automatically decrease the fluid and/or gaseous communication between a taste and/or smell modifying substance and a person's oral cavity and/or nasal cavity when the person digests a selected nutrient or food type within their mouth.

In an example, a flow control mechanism can automatically and selectively increase fluid and/or gaseous communication between a taste and/or smell modifying substance and a person's oral cavity and/or nasal cavity by the dissolution of a member that separates the substance from the person's oral cavity and/or nasal cavity. In an example, a chemical reaction can dissolve one or more members that separate the substance from a person's oral cavity and/or nasal cavity when the person digests a selected nutrient and/or food type. In an example, one or more members that block fluid and/or gaseous communication between the substance and the person's oral cavity and/or nasal cavity can be dissolved by a chemical reaction when these members are exposed to a selected nutrient or food.

In an example, fluid communication between a taste-modifying substance and a person's oral cavity can occur due to the movement of a member that limits fluid communication between the taste-modifying substance and a person's oral cavity, wherein this movement is caused by a chemical reaction to digestion of a selected nutrient or food type.

In an example, fluid communication between a taste-modifying substance and a person's oral cavity can occur due to osmotic exposure of the substance from an implanted reservoir to the person's oral cavity. In an example, fluid communication between a taste-modifying substance and a person's oral cavity can occur due when saliva flows from the person's oral cavity into an implanted reservoir. In an example, fluid communication between a taste-modifying substance and a person's oral cavity can occur due to osmosis of saliva from the person's oral cavity into an implanted reservoir.

In various examples, fluid and/or gaseous communication between a taste-modifying substance and a person's oral cavity can occur by one or more means selected from the group consisting of: dissolution or movement of a member between the taste-modifying substance and a person's oral cavity, wherein this dissolution or movement is a chemical reaction to digestion of a selected nutrient or food type in the person's mouth; active release, pumping, and/or spraying of the substance from an implanted reservoir into the person's oral cavity; osmosis of the substance from an implanted reservoir into the person's oral cavity and/or osmosis of saliva from the person's oral cavity into an implanted reservoir.

In various examples, a flow control mechanism can automatically increase the fluid and/or gaseous communication between a taste and/or smell modifying substance and a person's oral cavity and/or nasal cavity by using a peristaltic, osmotic, electromechanical, piezoelectric, biochemical, Micro Electrical Mechanical System (MEMS), or elastomeric pump to pump the substance from an implanted reservoir into the person's oral cavity and/or nasal cavity as a person digests a selected nutrient or food type within their mouth.

In various examples, a flow control mechanism can be programmed to automatically adjust the fluid and/or gaseous communication between a taste and/or smell modifying substance and a person's oral cavity and/or nasal cavity by a means selected from the group consisting of: adjusting a valve between an implanted reservoir (that contains the substance) and the person's oral cavity and/or nasal cavity; a chemical reaction that dissolves a member that separates the substance from the person's oral cavity and/or nasal cavity; spraying the substance from an implanted reservoir into the person's oral cavity and/or nasal cavity; and using a peristaltic, osmotic, electromechanical, piezoelectric, biochemical, MEMS, or elastomeric pump to pump the substance from an implanted reservoir into the person's oral cavity and/or nasal cavity.

In various examples, a flow control mechanism can be wirelessly programmed or otherwise adjusted by a remote control unit that is external to the person's body. In an example, a flow control mechanism can be programmed or adjusted by a remote control unit to change the timing of how a taste and/or smell modifying substance is brought into fluid and/or gaseous communication with the person's oral cavity and/or nasal cavity as a person digests a selected nutrient or food type within their mouth. In an example, a flow control mechanism can be programmed or adjusted by a remote control unit to change the amount of taste and/or smell modifying substance that is brought into fluid and/or gaseous communication with the person's oral cavity and/or nasal cavity as a person digests a selected nutrient or food type within their mouth.

In an example, a flow control mechanism can be programmed or otherwise adjusted to change which nutrients or foods trigger having a taste and/or smell modifying substance brought into fluid and/or gaseous communication with the person's oral cavity and/or nasal cavity. In an example, a flow control mechanism can be programmed or otherwise adjusted to change which taste and/or smell modifying substances are brought into fluid and/or gaseous communication with the person's oral cavity and/or nasal cavity in response to which nutrients or foods.

In an example, a flow control mechanism can be an electronic mechanism. In an example, a flow control mechanism may comprise a microchip or miniature electronic processor. In an example, a flow control mechanism can be powered by a rechargeable battery. In an example, a flow control mechanism can be powered by jaw movement. In an example, a flow control mechanism can be powered by glucose.

In an example, a flow control mechanism can automatically learn and change its programming based on the person's eating patterns and exercise patterns. In an example, this learning can occur within a microchip or electronic processor located within an implanted device. In an example, this learning can be done using a neural network. In an example, this learning can occur in a remote computer that is in wireless communication with the implanted device. In an example, a flow control mechanism can be a MEMS mechanism. In an example, a flow control mechanism can be a microfluidic mechanism.

In an example, a flow control mechanism can include electronic, chemical, biochemical, biological, digestive, and/or neurological components. In an example, a flow control mechanism can comprise enzymes or reagents that react to particular nutrients or to foods with high concentrations of those nutrients. In various examples, a flow control mechanism can be selected from the group consisting of: an electronic mechanism, a biochemical mechanism, a biological mechanism, an enzymatic mechanism, a MEMS mechanism, and a microfluidic mechanism.

In an example, a flow control mechanism can change how a taste and/or smell modifying substance is brought into fluid and/or gaseous communication with the person's oral cavity and/or nasal cavity based on the person's exercise patterns or caloric expenditure during a period of time. In an example, the control mechanism can allow the person to digest a limited amount of a selected nutrient or food type, during a period of time, before further digestion of that nutrient or food type causes the taste and/or smell modifying substance to be brought into fluid and/or gaseous communication with the person's oral cavity and/or nasal cavity.

In an example, a flow control mechanism can be programmed with allowable amounts of selected nutrients or types of food that can be consumed, during a period of time, and will only modify the taste and/or smell of those nutrients or types of food if the person consumes more than the allowable amounts during a period of time. In an example, the control mechanism can track the cumulative digestion of a selected nutrient or food type during a period of time and not modify the taste and/or smell of that nutrient or food type in a person's mouth unless the amount that is digested exceeds a selected amount.

In various examples, a flow control mechanism can be designed, programmed, or otherwise adjusted to selectively modify the taste and/or smell of selected nutrients or foods based on: the time of the day (to reduce snacking between meals or binge eating at night); the person's cumulative caloric expenditure (to reward exercise and achieve energy balance); special social events and holidays (to allow temporary relaxation of dietary restrictions); physical location measured by GPS (to discourage eating in locations that are associated with unhealthy consumption); and/or social networking connections and support groups (to provide peer support for willpower enhancement).

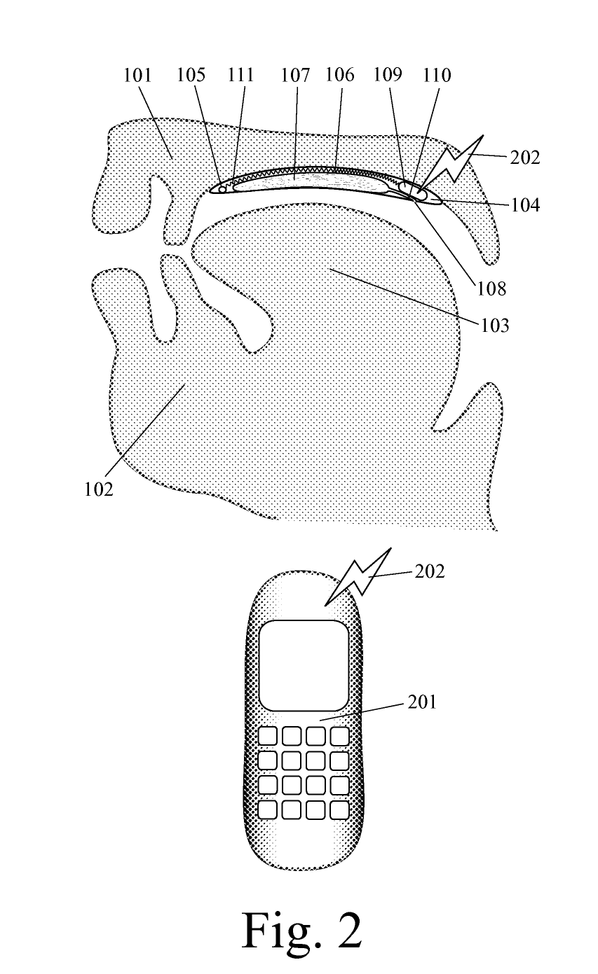

FIG. 2 shows an example of a taste and/or smell modifying implantable device that communicates wirelessly with an external control unit 201. In an example, electromagnetic signals 202 can be transmitted from external control unit 201 and received by flow control mechanism 109. In an example, external control unit 201 can be used to wirelessly program, or otherwise adjust the operation of, flow control mechanism 109. In an example, electromagnetic signals 202 can be transmitted from flow control mechanism 109 and received by external control unit 201.

In an example, external control unit 201 can have a touch-screen interface that allows the person to program, or otherwise adjust, the operation of flow control mechanism 109. In an example, external control unit can have a button or keyboard interface that allows the person to program, or otherwise adjust, the operation of flow control mechanism 109. In an example, external control unit can have a voice recognition interface that allows the person to program, or otherwise adjust, the operation of flow control mechanism 109. In an example, external control unit can have a gesture recognition interface that allows the person to program, or otherwise adjust, the operation of flow control mechanism 109. In an example, external control unit can have a holographic interface that allows the person to program, or otherwise adjust, the operation of flow control mechanism 109. In an example, for security purposes control unit 201 may require bio-identification of the user in order to program, or otherwise adjust, the operation of flow control mechanism 109.

In an example, signals from flow control mechanism 109 can provide diagnostic or dietary information that can be used as part of an overall system of energy balance and weight management. In an example, external control unit 201 can be in data communication with an internet website as part of an overall system of nutritional improvement, energy balance, and weight management. In an example, external control unit 201 can be in data communication with an social network as part of a support system for nutritional improvement, energy balance, and weight management. In an example, flow control mechanism 109 can be in wireless communication with a remote computer and/or the internet as part of a clinical or social support system to help the person to improve their eating habits and manage their weight. In an example, external control unit 201 can be in data communication with a provider of insurance or health benefits in order to provide financial rewards and incentives for proper nutrition, energy balance, and weight management.

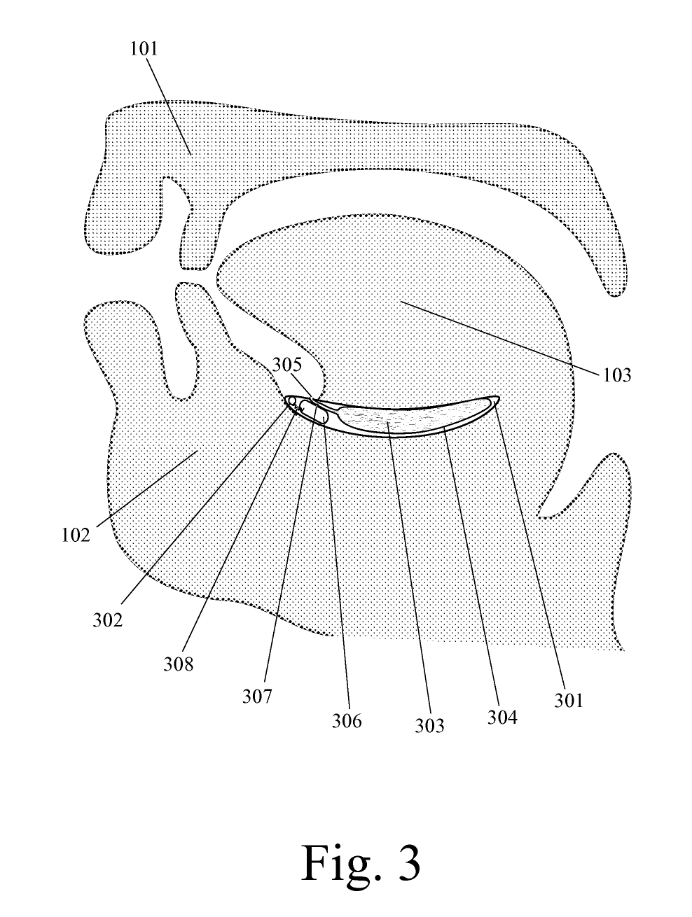

FIG. 3 shows an example of a taste and/or smell modifying device that is implanted into, or underneath, the person's tongue 103. In this example, an implanted and automatic taste-modification device comprises: a housing 301 which is implanted into, or under, the person's tongue 103; a nutrient or food sensor 302 in housing 301 which is in flowable communication with the person's oral cavity; a flowable taste and/or smell modifying substance 303; a substance reservoir 304 which contains the taste and/or smell modifying substance until it is released; an opening 305 between reservoir 304 and the person's oral cavity through which the taste and/or smell modifying substance 303 can flow; a flow control mechanism 306 which automatically controls the flow of substance 303 through opening 305; a valve 307 which is controlled by flow control mechanism 306; and a wire 308 which transmits signals from sensor 302 to flow control mechanism 306.

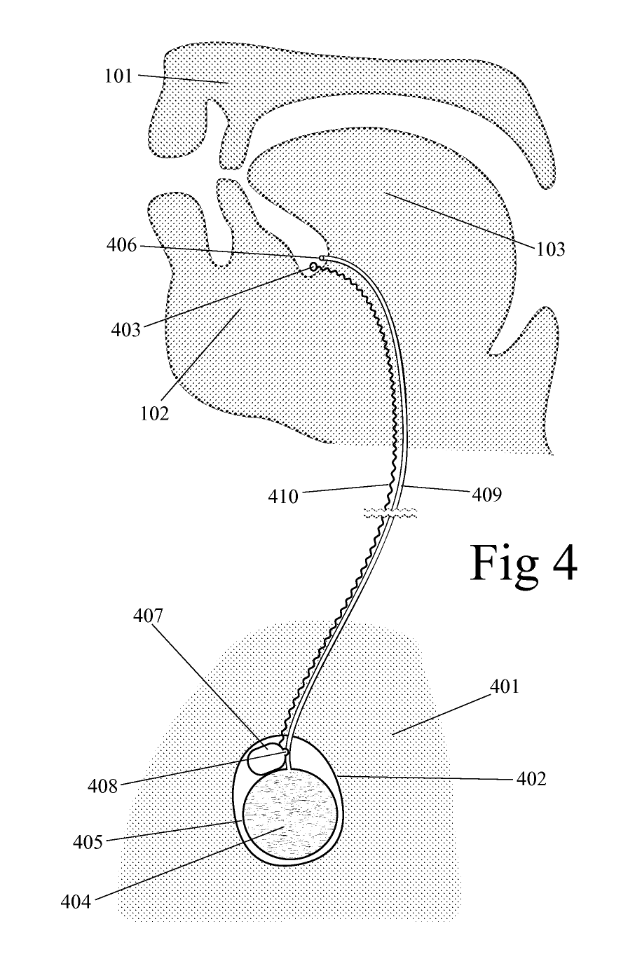

FIG. 4 shows an example of a taste and/or smell modifying device wherein the housing for a reservoir is implanted within the person's chest and/or abdominal cavity. In this example, even though the housing is not attached to, or implanted within, the person's oral cavity or nasal cavity, the reservoir is nonetheless in fluid communication with the person's oral cavity by means of a lumen. In this example, there is a nutrient or food sensor that is in fluid communication with the person's oral cavity and is connected to the flow control mechanism by a wire. In an example, having the main housing, reservoir, and flow control mechanism of the device be located away from (but still be connected to) the person's oral cavity can be less bothersome for the person and easier to implant.

In the example shown in FIG. 4, an implanted and automatic taste-modification device comprises: a housing 402 which is configured to be implanted within the person's chest and/or abdominal cavity 401; a nutrient or food sensor 403 which is in flowable communication with the person's oral cavity; a flowable taste and/or smell modifying substance 404; a substance reservoir 405 which contains taste and/or smell modifying substance 404 until the substance is released; an opening 406 on lumen 409 between reservoir 405 and the person's oral cavity through which taste and/or smell modifying substance 404 can flow; a flow control mechanism 407 which automatically controls the flow of substance 404 through lumen 409 and opening 406; a valve 408 which is controlled by flow control mechanism 407; and a wire 410 which transmits signals from sensor 403 to flow control mechanism 407. The dotted “zig-zag” lines across lumen 409 and wire 410 in the middle of FIG. 4 figure indicate an indefinite distance that is not necessarily represented by the dimensions of FIG. 4.

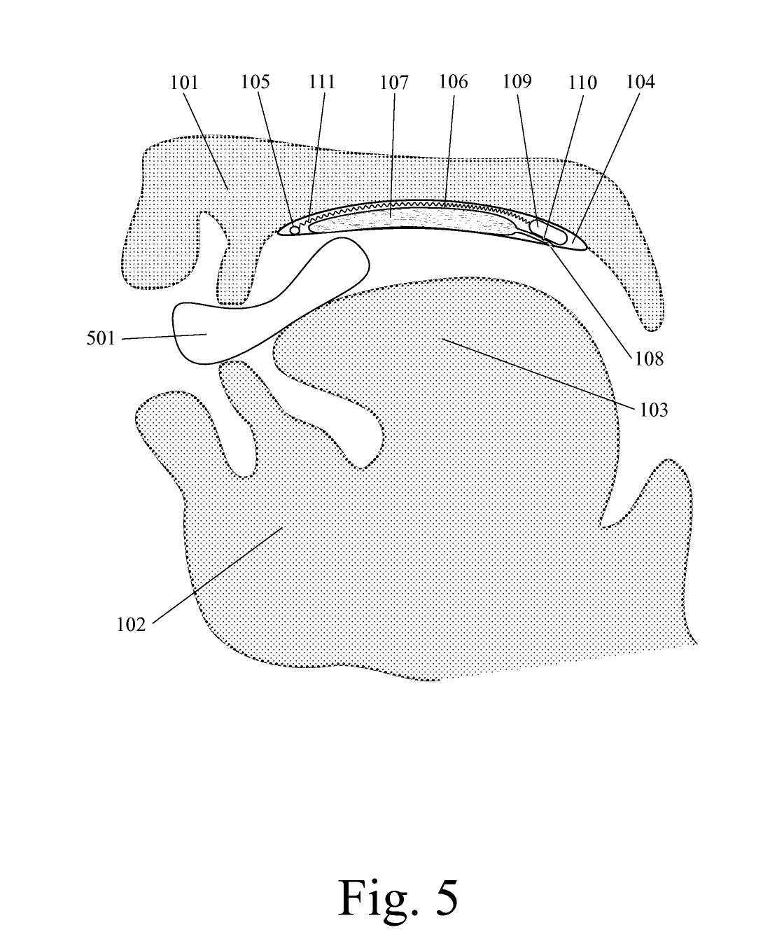

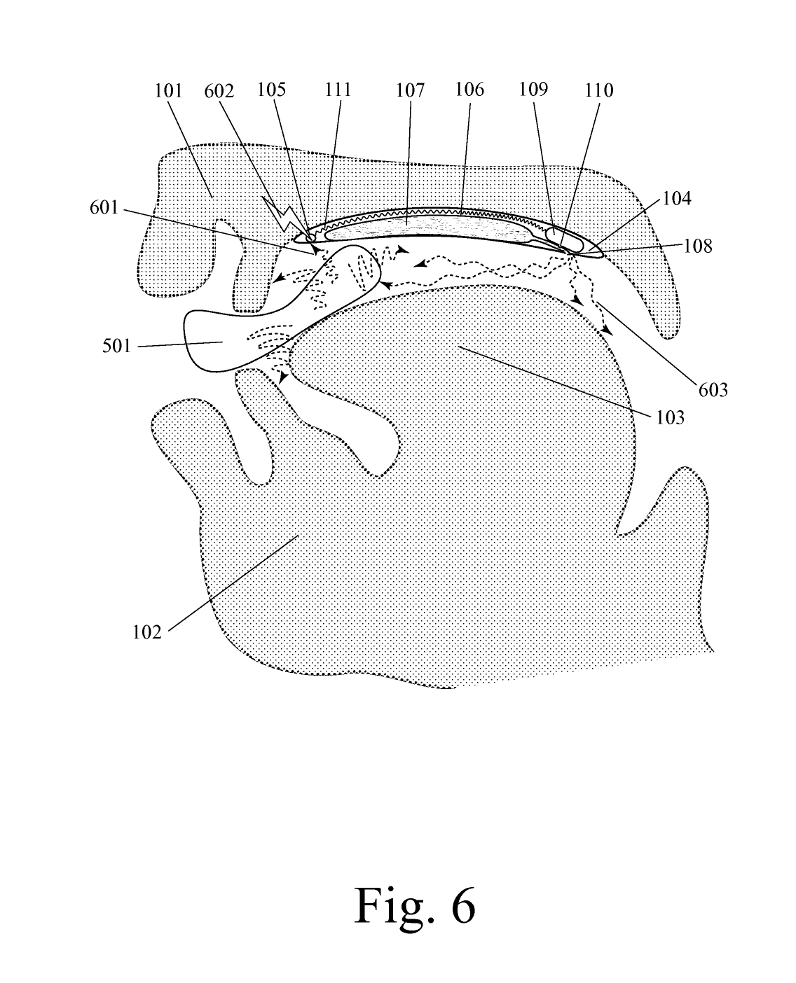

FIGS. 5 through 7 provide a three-figure sequence showing an example of how a device and method can use automatic taste-modification to change a person's eating habits. FIG. 5 shows the taste-modifying device that was introduced in FIG. 1 with the addition of a piece of food 501 that the person has inserted into their mouth. In FIG. 6, the digestion of food 501 has begun due to chewing action and contact between the food and saliva in the person's mouth. In FIG. 6, nutrients and chemicals 601 are released from food 501 into the person's oral cavity. In FIG. 6, these released nutrients and chemicals are represented by wavy dotted-line arrows 601.

FIG. 6 also shows that released nutrients and chemicals 601 have come into contact with nutrient or food sensor 105. In this example, nutrient or food sensor 105 is a chemical sensor. In this example, nutrient or food sensor 105 analyzes nutrients and chemicals 601 to identify food 501 as being one of the selected nutrients or foods for which the device is designed to trigger the release of taste and/or smell modifying substance 107. Accordingly, in FIG. 6, sensor 105 is shown sending an electric signal 602 concerning this identification to flow control mechanism 109 via wire 111.

Also in FIG. 6, in response to receiving signal 602, the flow control mechanism 109 opens valve 110. The opening of this valve releases taste and/or smell modifying substance 107 into the person's oral cavity while food 501 is still being digested. In another example, flow control mechanism 109 can activate a pump or spraying mechanism to release the taste and/or smell modifying substance instead of just opening a valve. The release of the taste and/or smell modifying substance into the person's oral cavity is represented in FIG. 6 by wavy dotted-line arrows 603. In this manner, the taste and/or smell modifying substance comes into contact with taste buds on the person's tongue 103. In another example, this substance can also come into contact with the person's olfactory receptors in their nasal cavity.

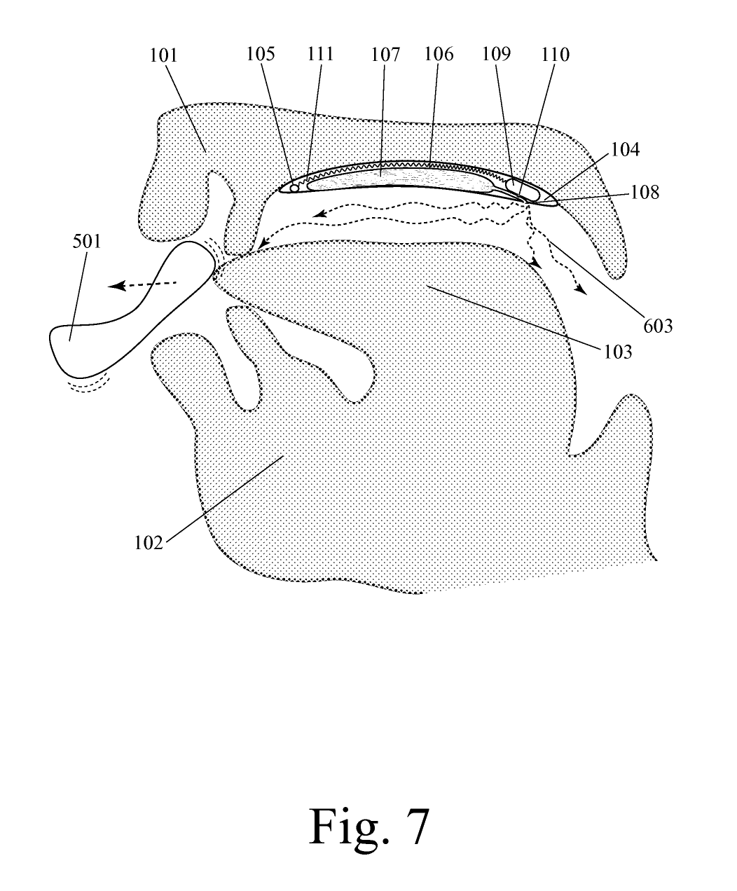

FIG. 7 shows the final step of this three-figure sequence. In FIG. 7, the person is shown as responding to contact between the taste-modifying substance and their taste buds by ejecting food 501 out of their mouth with their tongue 103. In this example, the taste-modifying substance has such a bad taste that it overpowers the normal flavor of food 501 and makes it unpalatable. In this manner, the device disclosed herein provides immediate and selective negative feedback in response to consumption of this particular food. This reduces consumption of this food.

FIGS. 1 through 7 show some examples of how this invention can be embodied in a device that modifies the taste and/or smell of a selected nutrient or food type as the nutrient or food is digested within a person's mouth, comprising: (a) a nutrient or food sensor that automatically and selectively detects when a person is digesting a selected nutrient or food type within the person's mouth, wherein this sensor is configured to be attached to or implanted within the person's body, and wherein this sensor is in fluid and/or gaseous communication with the person's oral cavity and/or nasal cavity; (b) a taste and/or smell modifying substance that modifies, masks, or blocks a taste and/or smell of food as food is digested within the person's mouth when the substance is brought into fluid and/or gaseous communication with the person's oral cavity and/or nasal cavity; (c) a substance reservoir in which a taste and/or smell modifying substance is stored until it is brought into fluid and/or gaseous communication with the person's oral cavity and/or nasal cavity, wherein this reservoir is configured to be attached to or implanted within the person's body; and (d) a flow control mechanism that automatically increases the fluid and/or gaseous communication between a taste and/or smell modifying substance and the person's oral cavity and/or nasal cavity when a nutrient or food sensor detects that the person is digesting a selected nutrient or food type within their mouth.

In an example, this invention can be embodied in an implantable device that automatically modifies the taste and/or smell of a selected nutrient or food as the nutrient or food is consumed, comprising: (a) a nutrient or food sensor that detects when a person is consuming a selected nutrient or food, wherein this sensor is configured to be attached to or implanted within the person's body, and wherein this sensor is in fluid and/or gaseous communication with the person's oral cavity and/or nasal cavity; (b) a taste and/or smell modifying substance that modifies the taste or smell of food when the substance is brought into fluid and/or gaseous communication with the person's oral cavity and/or nasal cavity as the food is consumed; (c) a substance reservoir in which the taste and/or smell modifying substance is stored until it is brought into fluid and/or gaseous communication with the person's oral cavity and/or nasal cavity, wherein this reservoir is configured to be attached to or implanted within the person's body; and (d) a flow control mechanism that automatically increases the fluid or gaseous communication between the taste and/or smell modifying substance and the person's oral cavity and/or nasal cavity when the sensor detects that the person is consuming a selected nutrient or food.

FIGS. 1 through 7 also show some examples of how this invention can be embodied in a method for automatically modifying a taste and/or smell of a selected nutrient or food as the nutrient or food is digested within a person's mouth, comprising: (a) storing a taste and/or smell modifying substance in a reservoir that is configured to be attached to or implanted within the person's body; (b) automatically detecting when a person is digesting a selected nutrient or food type within their mouth, wherein such detecting is done using a sensor that is in fluid and/or gaseous communication with the person's oral cavity and/or nasal cavity; and (c) automatically increasing fluid and/or gaseous communication between a taste and/or smell modifying substance and the person's oral cavity and/or nasal cavity as the person digests the selected nutrient or food type in their mouth in order to change the amount of the selected nutrient and/or food type that the person consumes.

In an example, this invention can be embodied in a method for automatically modifying the taste and/or smell of a selected nutrient or food type in a person's mouth if the person consumes more than a selected amount of the nutrient or food type: (a) storing a taste and/or smell modifying substance in a reservoir that is configured to be attached to or implanted within a person's body; (b) detecting when the person is digesting a selected nutrient or food type within their mouth; (c) allowing the person to digest a selected amount of the selected nutrient or food type, during a period of time, without modifying the taste and/or smell of the selected nutrient or food type; and (d) increasing fluid and/or gaseous communication between the taste and/or smell modifying substance and the person's oral cavity and/or nasal cavity if the person digests more than the selected amount of the selected nutrient or food type.

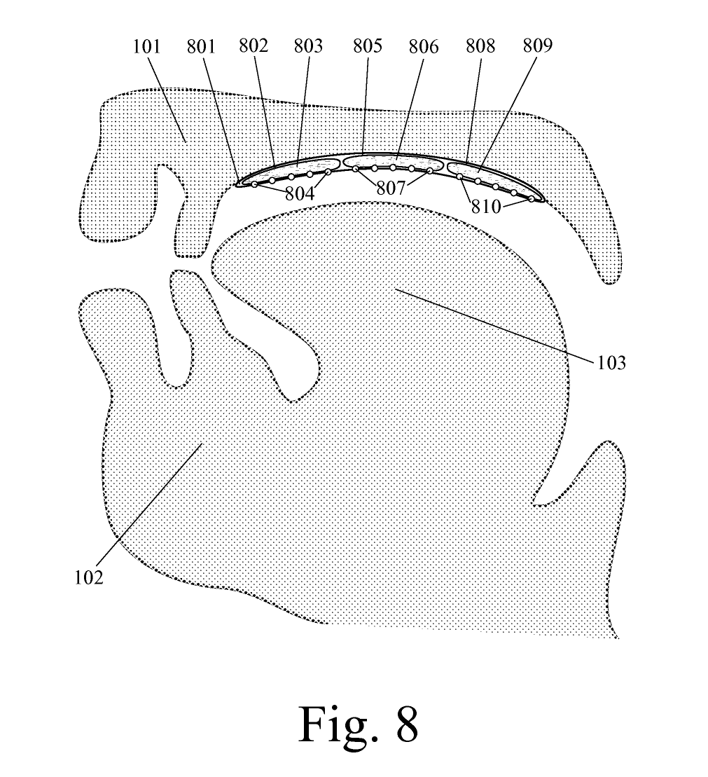

FIGS. 8 through 11 provide a four-figure sequence showing another example of how a device and method can use automatic taste-modification to change a person's eating habits.

FIG. 8 shows a cross-sectional figure of an implanted taste-modification device that is attached to, or implanted within, a person's palatal vault. This device in FIG. 8 includes housing 801 that is attached to the person's palatal vault with removable dental adhesive. In another example, this housing can be attached to the person's vault with adhesive tape or an adhesive patch. In this example, housing 801 is flexible, biocompatible, and lubricious so that it does not irritate the person's tongue.

Unlike the examples in previous figures, the example of this invention that is shown in FIG. 8 has multiple (three) substance reservoirs, 802, 805, and 808, instead of just one. Also, this example has multiple (three) different types of taste and/or smell modifying substances, 803, 806, and 809, instead of just one. In this example, there is a different taste and/or smell modifying substance in each of the different reservoirs. In another example, there could be different dosages of the same taste and/or smell modifying substance in each of the different reservoirs.

The embodiment of this invention that is shown in FIG. 8 also includes three sets of dissolvable plugs, 804, 807, and 810, that plug holes between the three reservoirs, 802, 805, and 808, and the person's oral cavity. As long as these plugs remain intact, they prevent the taste and/or smell modifying substances, 803, 806, and 809, from coming into fluid and/or gaseous communication with the person's oral cavity. In this example, each of these sets of plugs, 804, 807, and 810, can be dissolved by contact with a particular nutrient or food type.

When a set of plugs dissolves due to contact with a selected nutrient or food type, then the substance in the substance reservoir associated with that set comes into fluid and/or gaseous communication with the person's oral cavity. In an example, this fluid and/or gaseous communication can be primarily flow or osmosis of the substance out of the reservoir into the person's oral cavity. In an example, this fluid and/or gaseous communication can be primarily flow or osmosis of saliva from the person's oral cavity into the reservoir.

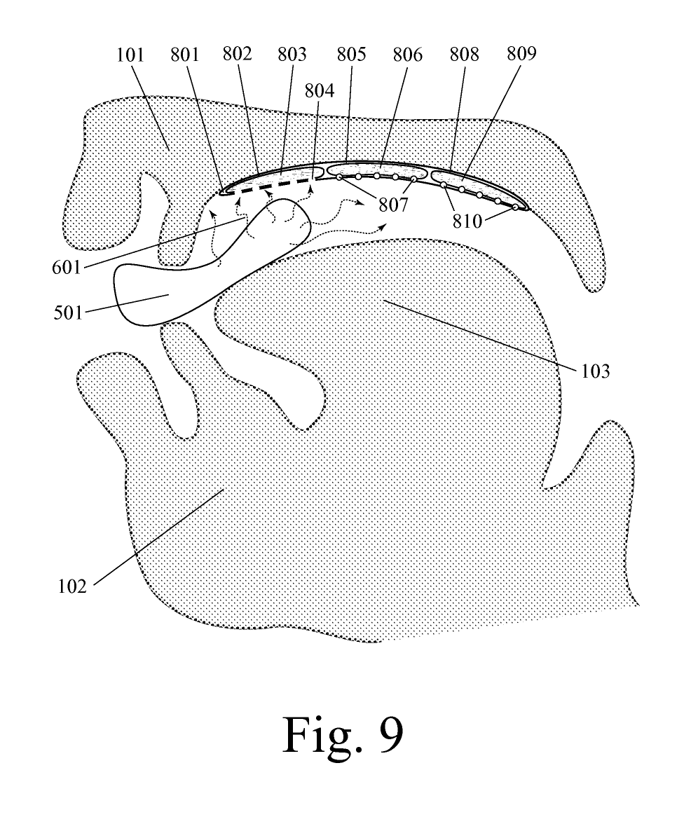

FIG. 9 shows the same taste-modification device that was introduced in FIG. 8, with the addition of a piece of food 501 that the person has inserted into their mouth. In FIG. 9, digestion of this piece of food 501 has started due to chewing and contact with saliva. In FIG. 9, nutrients and chemicals 601 are being released from digestion of piece of food 501. These nutrients and chemicals are represented in FIG. 9 by wavy dotted-line arrows 601.

FIG. 9 also shows that the nutrients and chemicals 601 that have been released by digestion of food 501 have reached and dissolved the first set of plugs 804 in the device. In an example, plugs 804 can be dissolved by a chemical reaction with the nutrients or chemicals 601 that are released by early digestion of piece of food 501. Plugs 804 are made of a safe and non-toxic material which does not harm the person when it is consumed. Dissolution of the first set of plugs 804, triggered by consumption of food 501, allows substance 803 to come into fluid and/or gaseous communication with the person's oral cavity. In this example, this communication occurs primarily by a flow of substance 803 out of reservoir 802 into the person's oral cavity.

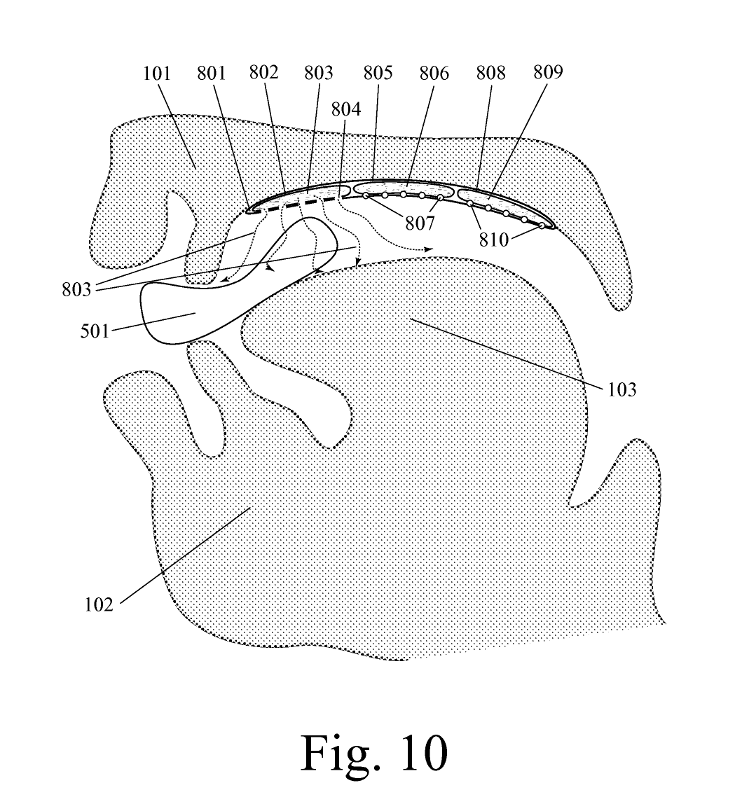

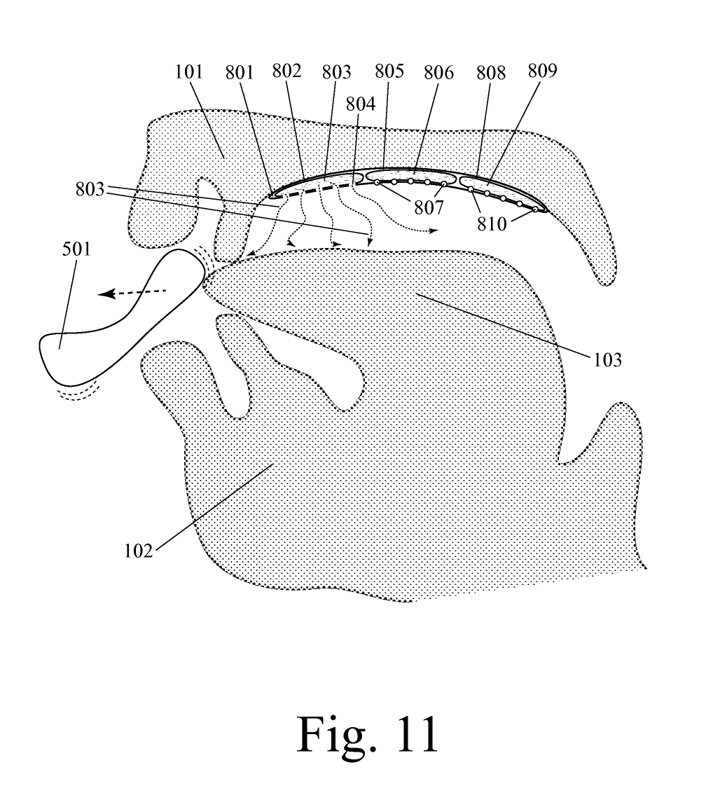

FIG. 10 shows the next step in this four-figure sequence of modification of food taste and consumption. In FIG. 10, taste and/or smell modifying substance 803 is leaking out of reservoir 802 because the first set of plugs 804 has been dissolved by contact with nutrients or chemicals from food 501. FIG. 10 shows substance 803 being dispersed throughout the person's mouth, such as in saliva, and coming into contact with taste buds on the person's tongue 103. FIG. 11 shows the final step in this four-figure sequence. In FIG. 11, the person is shown responding to the bad taste of substance 803 by ejecting piece of food 501 from their mouth with their tongue 103.

In the embodiment of this invention that is shown in FIGS. 8 through 11, the sets of dissolvable plugs, 804, 807, and 810, serve multiple roles. They serve to identify digestion of a particular type of nutrient or food and they also serve to directly release the taste and/or smell modifying substance into the person's oral cavity. In this example, dissolvable plugs serve a combined function as sensors and flow control mechanisms. Embodiments of this invention with dissolvable members, such as plugs, are more likely to be based on chemical mechanisms than electronic mechanisms. Both are within the scope of this automatic taste and/or smell modifying invention.

As shown in the embodiment of this invention in FIGS. 8 through 11, a housing can contain multiple reservoirs (or a reservoir with multiple compartments) that contain different substances. In an example, multiple reservoirs can contain separate doses of the same taste and/or smell modifying substance. In an example, the device and method disclosed herein can release different types or quantities of taste and/or smell modifying substances in response to digestion of different types of nutrients or foods.

FIGS. 8 through 11 show an example of how this invention can be embodied in an implantable device that modifies the taste and/or smell of a selected nutrient or food type as the nutrient or food is digested within a person's mouth, comprising: (a) a taste and/or smell modifying substance that modifies, masks, or blocks a taste and/or smell of food as food is digested within the person's mouth when the substance is brought into fluid and/or gaseous communication with the person's oral cavity and/or nasal cavity; (b) a substance reservoir in which a taste and/or smell modifying substance is stored until it is brought into fluid and/or gaseous communication with the person's oral cavity and/or nasal cavity, wherein this reservoir is configured to be attached to or implanted within the person's body; and (c) a flow control mechanism that automatically and selectively increases the fluid and/or gaseous communication between a taste and/or smell modifying substance and the person's oral cavity and/or nasal cavity when a selected nutrient or food type is being digested within the person's mouth.

Similarly, FIGS. 8 through 11 also show an example of how this invention can be embodied in an implantable device that automatically modifies the taste and/or smell of a selected nutrient or food as the nutrient or food is consumed, comprising: (a) a taste and/or smell modifying substance that modifies the taste or smell of food when the substance is brought into fluid and/or gaseous communication with the person's oral cavity and/or nasal cavity as the food is consumed; (b) a substance reservoir in which the taste and/or smell modifying substance is stored until it is brought into fluid and/or gaseous communication with the person's oral cavity and/or nasal cavity, wherein this reservoir is configured to be attached to or implanted within the person's body; and (c) a flow control mechanism that automatically increases the fluid or gaseous communication between the taste and/or smell modifying substance and the person's oral cavity and/or nasal cavity when the person consumes a selected nutrient or food.

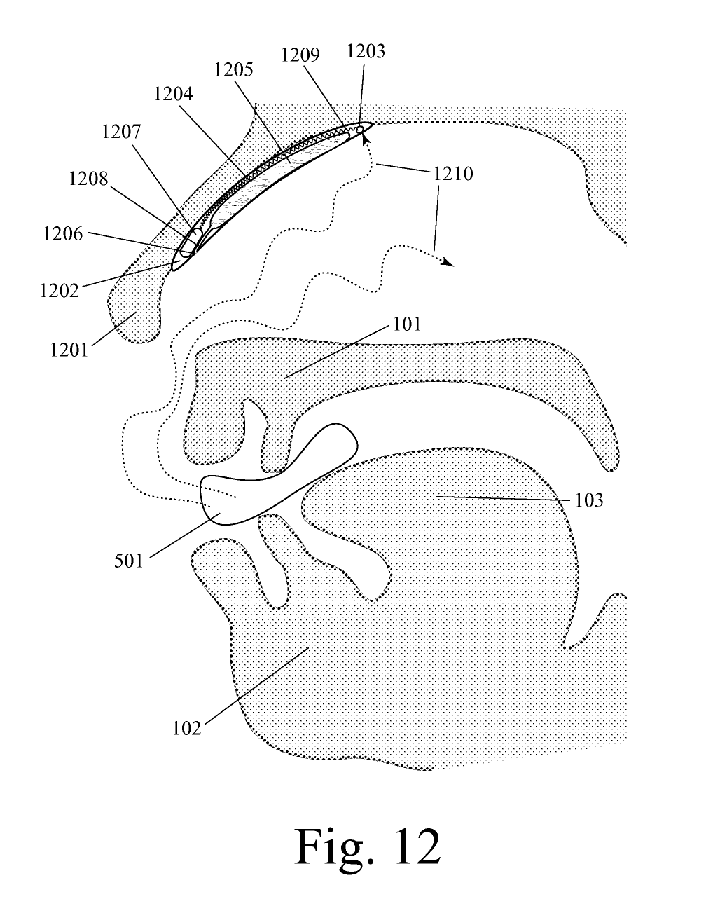

FIGS. 12 through 14 provide a three-figure sequence that shows the operation of an implanted device that is similar to the one shown in FIG. 1 except that it: is attached or implanted within the nasal cavity instead of the oral cavity; and releases a smell-modifying substance instead of a taste-modifying substance. Since smell has a very significant effect on taste perception and enjoyment of food, such a device can dramatically change a person's eating habits to help them to improve their nutrition and manage their weight.

FIG. 12 shows an example of a smell-modifying device that is attached to, or implanted within, a person's nasal cavity. In this example, the smell-modification device comprises: a housing 1202 which is implanted within the person's nose 1201; a nutrient or food sensor 1203 in housing 1202 which is in gaseous communication with the person's nasal cavity; a flowable smell modifying substance 1205; a substance reservoir 1204 which contains the smell-modifying substance until it is released; an opening 1206 between reservoir 1204 and the person's nasal cavity through which the smell-modifying substance 1205 can flow; a flow control mechanism 1207 which automatically controls the flow of substance 1205 through opening 1206; a valve 1208 which is controlled by flow control mechanism 1207; and a wire 1209 which transmits signals from sensor 1203 to flow control mechanism 1207.

FIG. 12 shows gaseous emissions (e.g. “smells”) 1210 coming from a piece of food 501 as it enters the person's mouth, the person starts to chew it, and digestion begins. These gaseous emissions are represented by wavy dotted-line arrows 1210. FIG. 12 shows these gaseous emissions entering the person's nasal cavity and being detected by nutrient or food sensor 1203.

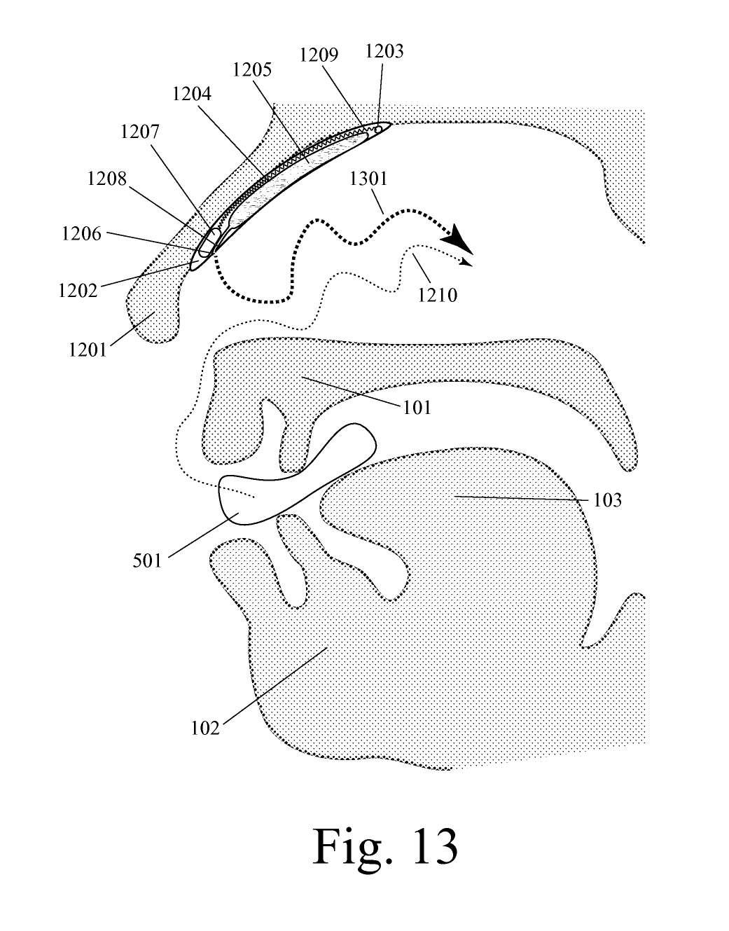

FIG. 13 shows the response of the device to these gaseous emissions. In FIG. 13, food 501 has been identified by nutrient or food sensor 1203 as one of the selected nutrients or foods to which the device should respond by releasing smell-modifying substance 1205. Nutrient or food sensor 1203 sends a signal to flow control mechanism 1207 which indicates that the person is starting to consume the selected nutrient or food type.

In response, flow control mechanism 1207 opens valve 1208 which releases smell-modifying substance 1205 into the person's nasal cavity. In another example, flow control mechanism 1207 can actively pump or spray a smell-modifying substance into the person's nasal cavity. The diffusion of the smell-modifying substance into the person's nasal cavity is represented in FIG. 13 by wavy dotted-line arrow 1301. In this example, the smell-modifying substance 1205 overpowers the normal smell 1210 of food 501. This overpowering is represented in FIG. 13 by having wavy dotted-line arrow 1301 be thicker than wavy dotted-line arrows 1210.

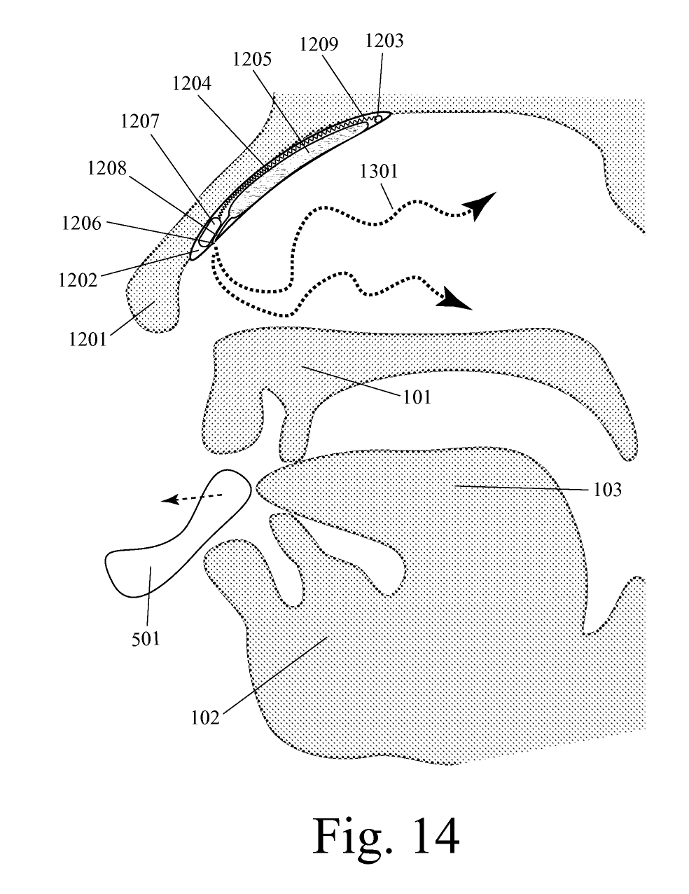

FIG. 14 shows the response of the person to the noxious smell of smell-modifying substance 1205 having been released 1301 into their nasal cavity. In this example, the smell is so unpleasant that it makes food 501 unappetizing. In this example, the person ejects food 501 out of their mouth with their tongue 103. In another example, the smell might not be so bad as to cause the person to eject the food out of their mouth, but it might be bad enough for the person to eat less of the food. In an example, this can cause moderate eating instead of overeating a food that is unhealthy to consume in excess. In an example, this can help to avoid binge eating. In an example, this can change a person's eating habits as part of an overall system for improved nutrition, energy balance, and weight management.

As shown in FIGS. 12 through 14, in an example, a flow control mechanism can automatically and selectively control the gaseous communication between a smell-modifying substance and a person's nasal cavity, wherein this control mechanism automatically and selectively changes this communication in response to the digestion of one or more selected nutrients, or types of food, within the person's mouth. In an example, fluid and/or gaseous communication between a smell-modifying substance and a person's nasal cavity can occur when the substance passively flows from an implanted reservoir into the person's nasal cavity. In an example, fluid and/or gaseous communication between a smell-modifying substance and a person's nasal cavity can occur when the substance is actively released from an implanted reservoir into the person's nasal cavity.

In an example, fluid and/or gaseous communication between a smell-modifying substance and a person's nasal cavity can occur when the substance is actively pumped from an implanted reservoir into the person's nasal cavity. In an example, fluid and/or gaseous communication between a smell-modifying substance and a person's nasal cavity can occur when the substance is actively sprayed from an implanted reservoir into the person's nasal cavity.

In an example, fluid and/or gaseous communication between a smell-modifying substance and a person's nasal cavity can occur when air from the nasal cavity is exposed to the substance within an implanted reservoir. In an example, fluid and/or gaseous communication between a smell-modifying substance and a person's nasal cavity can occur when air from the nasal cavity is actively circulated through an implanted reservoir that contains the substance. In an example, fluid and/or gaseous communication between a smell-modifying substance and a person's nasal cavity can occur when a valve is opened that causes some air of the air from a person's nasal cavity to flow through an implanted reservoir that contains the substance. In an example, a taste and/or smell modifying substance can be brought into gaseous communication with the person's nasal cavity by a flow of inhaled air from the person's nasal cavity into an implanted reservoir.

In various examples, fluid and/or gaseous communication between a smell-modifying substance and a person's nasal cavity can occur by one or more means selected from the group consisting of: the substance passively flowing from an implanted reservoir into the person's nasal cavity; the substance being actively pumped or sprayed from an implanted reservoir into the person's nasal cavity; air in the nasal cavity being passively exposed to the substance within an implanted reservoir; and air from the nasal cavity being actively circulated through an implanted reservoir that contains the substance.

When discussing possible variations on the embodiment of this device shown in FIG. 1, I discussed how a flow control mechanism 109 can actively pump the taste and/smell modifying substance 107 from reservoir 106 into the person's oral cavity. In an example, this pump can be a peristaltic pump. FIG. 15 shows, in greater detail, an example of how a peristaltic pump can be configured in this device and method. The lower part of FIG. 15 shows an example of a device that is similar to that shown in FIG. 1. The upper part of FIG. 15, connected to the lower part by dotted lines, shows an enlarged view of an oval portion of this lower part. The oval portion of the lower part is highlighted by a dotted-line oval. The upper portion of FIG. 15, with the enlarged oval portion of the lower part of the figure, shows greater detail concerning how a peristaltic pump can be incorporated into the device.

The upper part of FIG. 15 shows: housing 104; substance reservoir 106; smell and/or taste-modifying substance 107; opening 108; flow control mechanism 109; wire 111; flexible tube 1501 that connects reservoir 106 to opening 108; and rotating disk 1502 with four bulbous protrusions. When rotating disk 1502 is rotated by the flow control mechanism 109, its four bulbous protrusions squeeze the taste and/or smell modifying substance 107 through flexible tube 1501 and into the person's oral cavity. Rotating disk 1502 and flexible tube 1501 show one example of how a peristaltic pump can be used to release a taste and/or smell modifying substance into the person's oral cavity.

In this example, a peristaltic pump is used. In various examples, a flow control mechanism can automatically increase the fluid and/or gaseous communication between a taste and/or smell modifying substance and a person's oral cavity and/or nasal cavity by using a peristaltic, osmotic, electromechanical, piezoelectric, biochemical, Micro Electrical Mechanical System (MEMS), or elastomeric pump. The selected pump can pump the substance from an implanted reservoir into the person's oral cavity and/or nasal cavity as a person digests a selected nutrient or food type within their mouth.

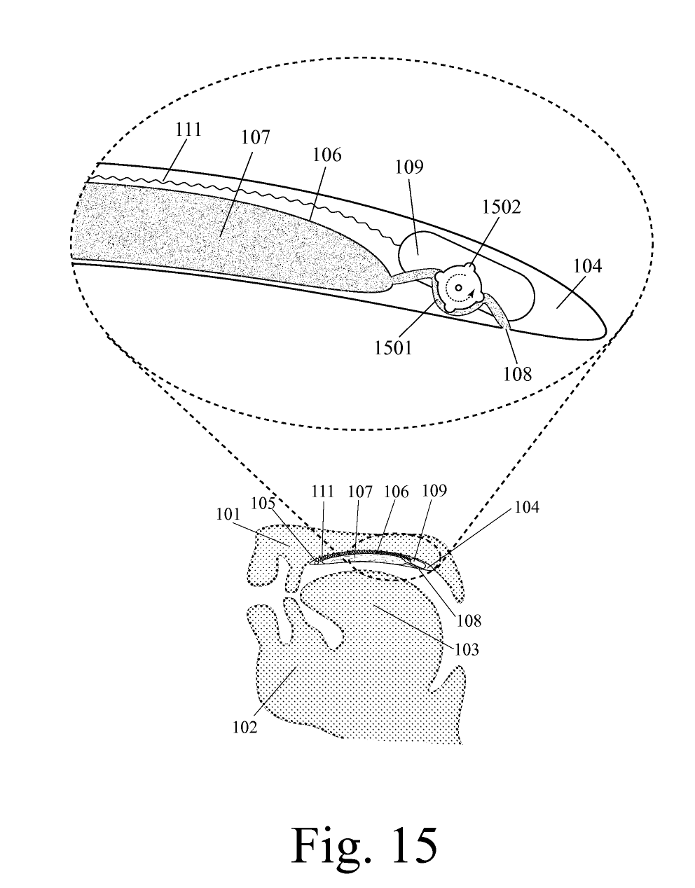

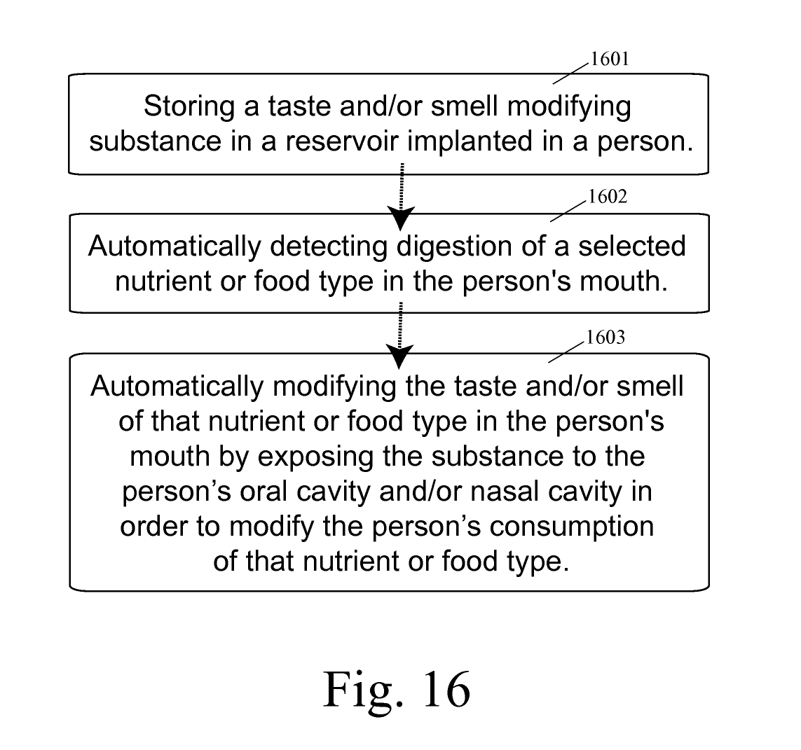

FIG. 16 shows an example of how this invention can be embodied in a method to modify the smell and/or taste of a selected nutrient or food while food is being digested in a person's mouth. This method can be part of an overall system for improving nutrition, energy balance, and weight management.

FIG. 16 shows a three-step method for taste and/or smell modification comprising: 1601 storing a taste and/or smell modifying substance in a reservoir implanted in a person; 1602 automatically detecting digestion of a selected nutrient or food type in the person's mouth; and 1603 automatically modifying the taste and/or smell of that nutrient or food type in the person's mouth by exposing the substance to the person's oral cavity and/or nasal cavity in order to modify the person's consumption. In an example, one or more of these steps can be performed in a flow control mechanism. In an example, this flow control mechanism can comprise an electronic or chemical mechanism.

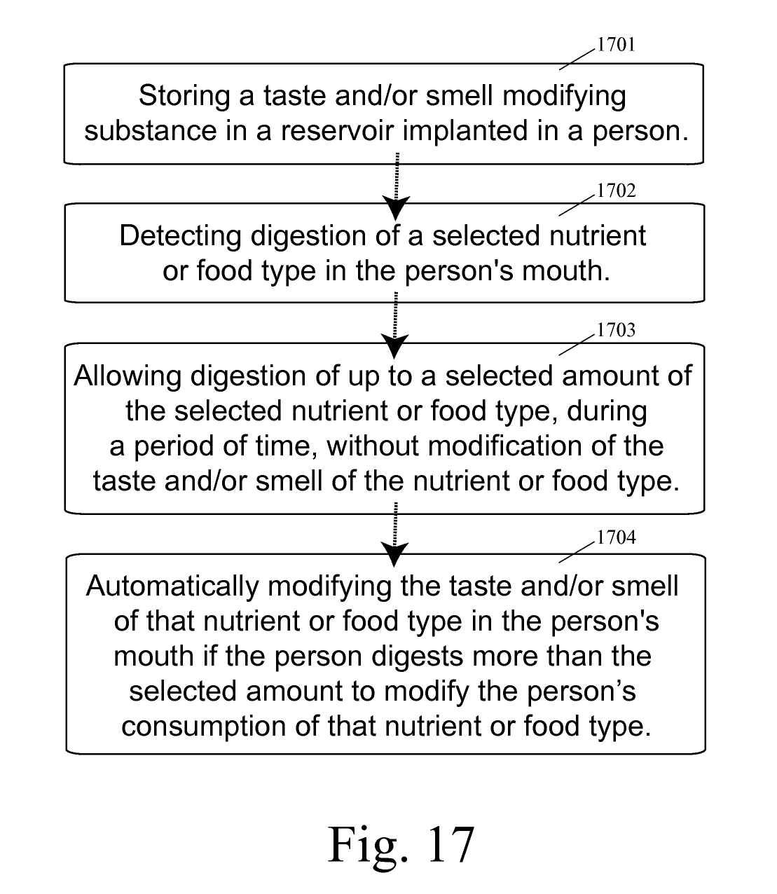

FIG. 17 shows another example of how this invention can be embodied in a method to modify the smell and/or taste of a selected nutrient or food while food is being digested in a person's mouth. This method can be part of an overall system for improving nutrition, energy balance, and weight management.

FIG. 17 shows a four-step method for taste and/or smell modification comprising: 1701 storing a taste and/or smell modifying substance in a reservoir implanted in a person; 1702 detecting digestion of a selected nutrient or food type in the person's mouth; 1703 allowing digestion of up to a selected amount of the selected nutrient or food type, during a period of time, without modification of the taste and/or smell of the nutrient or food type; and 1704 automatically modifying the taste and/or smell of that nutrient or food type in the person's mouth if the person digests more than the selected amount to modify the person's consumption of that nutrient or food type. In an example, one or more of these steps can be performed in a flow control mechanism. In an example, this flow control mechanism can comprise an electronic or chemical mechanism.

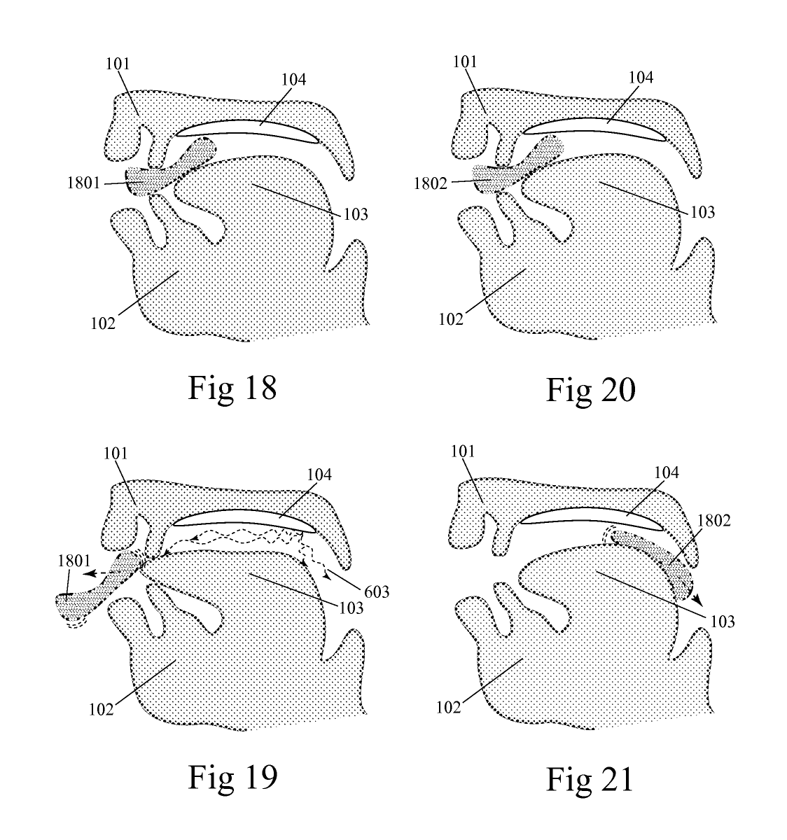

FIGS. 18 through 21 show, in greater detail, how the embodiment of this invention that was introduced in FIG. 1 can be used to selectively modify the taste of only a selected nutrient or food. In an example, FIGS. 18 through 21 show how this embodiment can discourage consumption of food that is designated (and detected) as unhealthy, without discouraging consumption of food that is designated as healthy. In an example, an unhealthy type of nutrient or food may be identified as being in the group consisting of: fried or deep-fried food, French fries, cholesterol or high-cholesterol food, fat or high-fat food or high-saturated-fat food, high-fructose corn syrup, salt or high-sodium food, simple or refined sugar or high-sugar food, hydrogenated oil, and soda pop.

Due to the smaller scale of FIGS. 18 through 21, only the housing 104 of the device is shown in FIGS. 18 through 21. The interior components shown in FIG. 1 are assumed to still be within housing 104, but housing 104 is shown as opaque because showing the interior components would unduly clutter these smaller-scale diagrams.

FIGS. 18 and 19 show what happens when a person eats a piece of unhealthy food. FIG. 18 shows the person starting to consume a piece of unhealthy food 1801. In FIG. 19, initial digestion of unhealthy piece of food 1801 has triggered the release of a bad-tasting substance 603 from device housing 104. As also shown in FIG. 19, this bad tasting substance 603 causes the person to eject unhealthy piece of food 1801 from their mouth. In an example, unhealthy food is detected by a chemical sensor within housing 104. FIGS. 18 through 19 show how this device can selectively discourage consumption of unhealthy food.

FIGS. 20 through 21 show what happens when a person eats a piece of healthy food. FIG. 20 shows the same device as shown in FIG. 18, except that now the person is starting to consume a piece of healthy food 1802. As shown in FIG. 21, digestion of healthy food 1802 does not trigger the release of bad tasting substance 603 from the device housing 104. In an example, the sensor can differentiate between selected (unhealthy) vs. non-selected (healthy) food. Healthy piece of food 1802 does not trigger the sensor or release bad-tasting substance 603. FIG. 21 shows that the person fully ingests healthy piece of food 1802 in an unhindered manner. Healthy piece of food 1802 is shown sliding down the person's throat into the rest of the digestive tract.

FIGS. 18 through 21 further illustrate the ability of this device and method to selectively modify the taste and/or smell of a selected (unhealthy) nutrient or food, while allowing the taste and/or smell of other (healthy) food to be remain unmodified. In an example, the designation of which foods will have their taste and/or smell modified is controlled by the flow control mechanism of this invention. In an example, the designation of which foods will have their taste and/or smell modified can be remotely adjusted and programmed by the person or a health care professional. This is a significant improvement over consumption-reducing devices in the prior art that are blind concerning what type of food is being consumed and thus reduce consumption (or absorption) of healthy food as well as unhealthy food.

FIGS. 1 through 21 show how this invention can be embodied in an implanted device that modifies the taste and/or smell of a selected nutrient or food type as the nutrient or food is digested within a person's mouth, comprising: a taste and/or smell modifying substance that modifies, masks, or blocks the taste and/or smell of food as food is digested within the person's mouth when the substance is brought into fluid and/or gaseous communication with the person's oral cavity and/or nasal cavity; a substance reservoir in which the taste and/or smell modifying substance is stored until it is brought into fluid and/or gaseous communication with the person's oral cavity and/or nasal cavity, wherein this reservoir is configured to be attached to or implanted within the person's body; and a flow control mechanism that can automatically and selectively increase the fluid and/or gaseous communication between the taste and/or smell modifying substance and the person's oral cavity and/or nasal cavity when a selected nutrient or food type is being digested within the person's mouth.

FIGS. 1 through 21 also show how this invention can be embodied in an implanted device that modifies the taste and/or smell of a selected nutrient or food type as the nutrient or food is digested within a person's mouth, comprising: a nutrient or food sensor that automatically and selectively detects when a person is digesting a selected nutrient or food type within the person's mouth, wherein this sensor is configured to be attached to or implanted within the person's body, and wherein this sensor is in fluid and/or gaseous communication with the person's oral cavity and/or nasal cavity; a taste and/or smell modifying substance that modifies, masks, or blocks the taste and/or smell of food as food is digested within the person's mouth when the substance is brought into fluid and/or gaseous communication with the person's oral cavity and/or nasal cavity; a substance reservoir in which the taste and/or smell modifying substance is stored until it is brought into fluid and/or gaseous communication with the person's oral cavity and/or nasal cavity, wherein this reservoir is configured to be attached to or implanted within the person's body; and a flow control mechanism that can automatically increase the fluid and/or gaseous communication between the taste and/or smell modifying substance and the person's oral cavity and/or nasal cavity when the nutrient or food sensor detects that the person is digesting a selected nutrient or food type within their mouth.

FIGS. 1 through 21 show how this invention can be embodied in a method for automatically and selectively modifying the taste and/or smell of a selected nutrient or food as the nutrient or food is digested within a person's mouth, comprising: storing a taste and/or smell modifying substance in a reservoir that is configured to be attached to or implanted within a person's body; automatically detecting when a person is digesting a selected nutrient or food type within their mouth; and automatically increasing fluid and/or gaseous communication between the taste and/or smell modifying substance and the person's oral cavity and/or nasal cavity as the person digests the selected nutrient or food type in their mouth.

FIGS. 1 through 21 also show how this invention can be embodied in a method for automatically and selectively modifying the taste and/or smell of a selected nutrient or food type in a person's mouth if the person consumes more than a selected amount of the nutrient or food type: storing a taste and/or smell modifying substance in a reservoir that is configured to be attached to or implanted within a person's body; detecting when the person is digesting a selected nutrient or food type within their mouth; allowing the person to digest a selected amount of the selected nutrient or food type, during a period of time, without modifying the taste and/or smell of the selected nutrient or food type; and modifying the taste and/or smell of the selected nutrient or food type in the person's mouth if the person digests more than the selected amount of the selected nutrient or food type.

In various examples, a nutrient or food sensor can be selected from the group consisting of: biochemical sensor, biological sensor, chemical-based sensor, cholesterol-based sensor, chromatography-based sensor, enzyme-based sensor, fat sensor, filtration-based sensor, glucose sensor, interferometer-based sensor, laboratory on a chip, membrane-based sensor, Micro Electrical Mechanical System (MEMS) sensor, microfluidic sensor, nanoparticle-based sensor, nanoscale sensor, micronutrient sensor, neural sensor, electromagnetic sensor, optics-based sensor, infrared-based sensor, protein-based sensor, reagent-based sensor, and strain sensor.

In various examples, a nutrient or food sensor can automatically and selectively detect digestion of a selected nutrient or food type in the person's mouth wherein this nutrient or food type is selected from the group consisting of: sugar or simple carbohydrates in general, saturated fat or fats in general, cholesterol, food with a high amount of sugar or simple carbohydrates in general, food with a high amount of saturated fat or fats in general, and high-cholesterol food.

In various examples, a taste and/or smell modifying substance can be selected so that a person eats less of a food when the substance comes into fluid and/or gaseous communication with the person's oral cavity and/or nasal cavity while the food is being digested in the person's mouth. In various examples, a taste and/or smell modifying substance can have a taste and/or smell that is bitter, sour, spicy, hot, or noxious. In various examples, a taste and/or smell modifying substance can be selected to have a high rating on a taste intensity scale so that it overpowers or masks the normal taste and/or smell of a selected food. In various examples, a taste and/or smell modifying substance can cause temporary ageusia or anosmia. In various examples, a taste and/or smell modifying substance can be Generally Recognized As Safe (GRAS) under Section 201(s) and Section 409 of the Federal Food, Drug, and Cosmetic Act.

In various examples, the fluid communication between the taste and/or smell modifying substance and the person's oral cavity can occur due to movement of the substance from the reservoir into the person's oral cavity and/or movement of saliva from the person's oral cavity into the reservoir. In various examples, a flow control mechanism can automatically increase the fluid and/or gaseous communication between the taste and/or smell modifying substance and the person's oral cavity and/or nasal cavity by opening a valve, or increasing the size of an opening, between the reservoir and the person's oral cavity and/or nasal cavity when a person digests a selected nutrient or food type within their mouth.

In various examples, a flow control mechanism can automatically increase the fluid and/or gaseous communication between the taste and/or smell modifying substance and the person's oral cavity and/or nasal cavity by pumping or spraying the substance from the reservoir into the person's oral cavity and/or nasal cavity when a person digests a selected nutrient or food type within their mouth.

In various examples, a flow control mechanism can be wirelessly programmed by a remote control unit that is external to the person's body in order to change the manner in which one or more taste and/or smell modifying substances are brought into fluid and/or gaseous communication with the person's oral cavity and/or nasal cavity in response to digestion of one or more selected nutrients or types of food.

In various examples, a flow control mechanism can change how the taste and/or smell modifying substance is brought into fluid and/or gaseous communication with the person's oral cavity and/or nasal cavity based on the person's eating patterns or caloric intake during a period of time. In various examples, a flow control mechanism can allow the person to digest a limited amount of a selected nutrient or food type, during a period of time, before further digestion of that nutrient or food type causes the taste and/or smell modifying substance to be brought into fluid and/or gaseous communication with the person's oral cavity and/or nasal cavity.

In various examples, a flow control mechanism can change how the taste and/or smell modifying substance is brought into fluid and/or gaseous communication with the person's oral cavity and/or nasal cavity based on the person's exercise patterns or caloric expenditure during a period of time. In various examples, a flow control mechanism can be part of an overall system for personal energy balance and weight management.

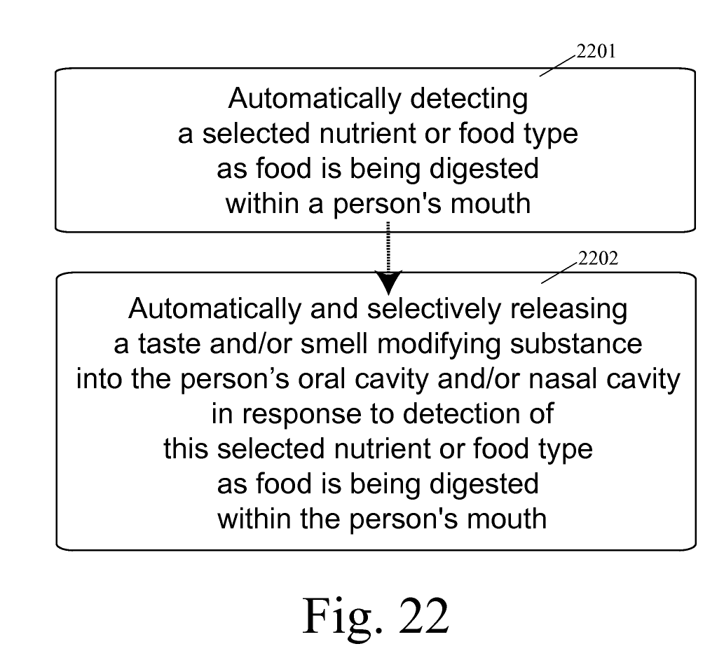

FIG. 22 shows another example of how this invention can be embodied in a method for modifying the taste and/or smell of one or more selected nutrient or food types while food is being digested in a person's mouth. Such taste and/or smell modification can reduce consumption of unhealthy food (and/or increase consumption of healthy food) as part of an overall system for improving nutrition, managing energy balance, and weight reduction. The method shown in FIG. 22 has two steps: 2201 automatically detecting a selected nutrient or food type as food is being digested within a person's mouth; and 2202 automatically and selectively releasing a taste and/or smell modifying substance into the person's oral cavity and/or nasal cavity in response to detection of this selected nutrient or food type as food is being digested within the person's mouth.

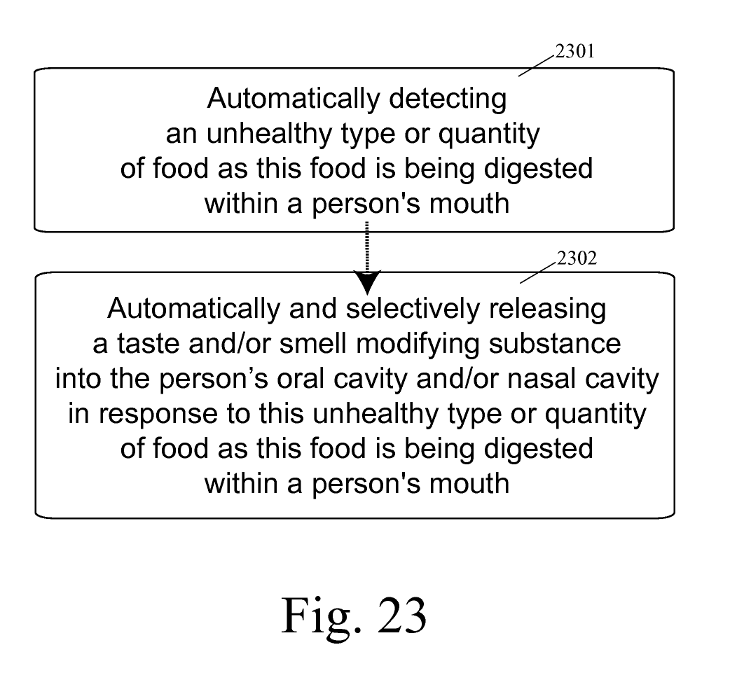

FIG. 23 shows an example of how this invention can be embodied in a method for modifying the taste and/or smell of an unhealthy type or quantity of food, as this food is being digested in a person's mouth. The method shown in FIG. 23 has two steps: 2301 automatically detecting an unhealthy type or quantity of food as this food is being digested within a person's mouth; and 2301 automatically and selectively releasing a taste and/or smell modifying substance into the person's oral cavity and/or nasal cavity in response to this unhealthy type or quantity of food as this food is being digested within a person's mouth.

In an example, an unhealthy type of nutrient or food may be identified as being in the group consisting of: fried or deep-fried food, French fries, cholesterol or high-cholesterol food, fat or high-fat food or high-saturated-fat food, high-fructose corn syrup, salt or high-sodium food, simple or refined sugar or high-sugar food, hydrogenated oil, and soda pop. In an example, an unhealthy quantity of food may be identified by the duration or concentration of such food that is detected in a person's mouth during a selected period of time.

In an example, the taste and/or smell of unhealthy food is modified to reduce consumption, but the taste and/or smell of healthy food is not modified. In an example, the taste and/or smell of moderate amounts or concentrations of unhealthy nutrients or foods are not modified, but the taste and/or smell of such unhealthy nutrients or foods are modified when the amount, duration, or concentration of such nutrients or foods in a person's mouth exceeds selected parameters.

I claim:

1. An implanted device that modifies the taste and/or smell of a selected nutrient or food type as the nutrient or food is digested within a person's mouth, comprising:
a taste and/or smell modifying substance that modifies, masks, or blocks the taste and/or smell of food as food is digested within the person's mouth when the substance is brought into fluid and/or gaseous communication with the person's oral cavity and/or nasal cavity;
a substance reservoir in which the taste and/or smell modifying substance is stored until it is brought into fluid and/or gaseous communication with the person's oral cavity and/or nasal cavity, wherein this reservoir is configured to be attached to or implanted within the person's body; and
a flow control mechanism that can automatically and selectively increase the fluid and/or gaseous communication between the taste and/or smell modifying substance and the person's oral cavity and/or nasal cavity when a selected nutrient or food type is being digested within the person's mouth.

2. An implanted device that modifies the taste and/or smell of a selected nutrient or food type as the nutrient or food is digested within a person's mouth, comprising:
a nutrient or food sensor that automatically and selectively detects when a person is digesting a selected nutrient or food type within the person's mouth, wherein this sensor is configured to be attached to or implanted within the person's body, and wherein this sensor is in fluid and/or gaseous communication with the person's oral cavity and/or nasal cavity;
a taste and/or smell modifying substance that modifies, masks, or blocks the taste and/or smell of food as food is digested within the person's mouth when the substance is brought into fluid and/or gaseous communication with the person's oral cavity and/or nasal cavity;
a substance reservoir in which the taste and/or smell modifying substance is stored until it is brought into fluid and/or gaseous communication with the person's oral cavity and/or nasal cavity, wherein this reservoir is configured to be attached to or implanted within the person's body; and
a flow control mechanism that can automatically increase the fluid and/or gaseous communication between the taste and/or smell modifying substance and the person's oral cavity and/or nasal cavity when the nutrient or food sensor detects that the person is digesting a selected nutrient or food type within their mouth.

3. The device in claim 2 wherein the nutrient or food sensor is selected from the group consisting of: biochemical sensor, biological sensor, chemical-based sensor, cholesterol-based sensor, chromatography-based sensor, enzyme-based sensor, fat sensor, filtration-based sensor, glucose sensor, interferometer-based sensor, laboratory on a chip, membrane-based sensor, Micro Electrical Mechanical System (MEMS) sensor, microfluidic sensor, nanoparticle-based sensor, nanoscale sensor, micronutrient sensor, neural sensor, electromagnetic sensor, optics-based sensor, infrared-based sensor, protein-based sensor, reagent-based sensor, and strain sensor.

4. The device in claim 2 wherein the nutrient or food sensor automatically and selectively detects digestion of a selected nutrient or food type in the person's mouth wherein this nutrient or food type is selected from the group consisting of: sugar or simple carbohydrates in general, saturated fat or fats in general, cholesterol, food with a high amount of sugar or simple carbohydrates in general, food with a high amount of saturated fat or fats in general, and high-cholesterol food.

5. The device in claim 2 wherein the taste and/or smell modifying substance is selected so that a person eats less of a food when the substance comes into fluid and/or gaseous communication with the person's oral cavity and/or nasal cavity while the food is being digested in the person's mouth.

6. The device in claim 2 wherein the taste and/or smell modifying substance has a taste and/or smell that is bitter, sour, spicy, hot, or noxious.

7. The device in claim 2 wherein the taste and/or smell modifying substance is selected to have a high rating on a taste intensity scale so that it overpowers or masks the normal taste and/or smell of a selected food.

8. The device in claim 2 wherein the taste and/or smell modifying substance causes temporary ageusia.

9. The device in claim 2 wherein the taste and/or smell modifying substance causes temporary anosmia.

10. The device in claim 2 wherein the taste and/or smell modifying substance is Generally Recognized As Safe (GRAS) under Section 201(s) and Section 409 of the Federal Food, Drug, and Cosmetic Act.

11. The device in claim 2 wherein fluid communication between the taste and/or smell modifying substance and the person's oral cavity occurs due to movement of the substance from the reservoir into the person's oral cavity and/or movement of saliva from the person's oral cavity into the reservoir.

12. The device in claim 2 wherein the flow control mechanism automatically increases the fluid and/or gaseous communication between the taste and/or smell modifying substance and the person's oral cavity and/or nasal cavity by opening a valve, or increasing the size of an opening, between the reservoir and the person's oral cavity and/or nasal cavity when a person digests a selected nutrient or food type within their mouth.

13. The device in claim 2 wherein the flow control mechanism automatically increases the fluid and/or gaseous communication between the taste and/or smell modifying substance and the person's oral cavity and/or nasal cavity by pumping or spraying the substance from the reservoir into the person's oral cavity and/or nasal cavity when a person digests a selected nutrient or food type within their mouth.

14. The device in claim 2 wherein the flow control mechanism changes how the taste and/or smell modifying substance is brought into fluid and/or gaseous communication with the person's oral cavity and/or nasal cavity based on the person's eating patterns or caloric intake during a period of time.

15. The device in claim 2 wherein the flow control mechanism allows the person to digest a limited amount of a selected nutrient or food type, during a period of time, before further digestion of that nutrient or food type causes the taste and/or smell modifying substance to be brought into fluid and/or gaseous communication with the person's oral cavity and/or nasal cavity.

16. The device in claim 2 wherein the flow control mechanism changes how the taste and/or smell modifying substance is brought into fluid and/or gaseous communication with the person's oral cavity and/or nasal cavity based on the person's exercise patterns or caloric expenditure during a period of time.

17. The device in claim 2 wherein the flow control mechanism can be wirelessly programmed by a remote control unit that is external to the person's body in order to change the manner in which one or more taste and/or smell modifying substances are brought into fluid and/or gaseous communication with the person's oral cavity and/or nasal cavity in response to digestion of one or more selected nutrients or types of food.

18. The device in claim 2 wherein the flow control mechanism is part of an overall system for personal energy balance and weight management.

19. A method for automatically and selectively modifying the taste and/or smell of a selected nutrient or food as the nutrient or food is digested within a person's mouth, comprising:
storing a taste and/or smell modifying substance in a reservoir that is configured to be attached to or implanted within a person's body;
automatically detecting when a person is digesting a selected nutrient or food type within their mouth; and
automatically increasing fluid and/or gaseous communication between the taste and/or smell modifying substance and the person's oral cavity and/or nasal cavity as the person digests the selected nutrient or food type in their mouth.

20. A method for automatically and selectively modifying the taste and/or smell of a selected nutrient or food type in a person's mouth if the person consumes more than a selected amount of the nutrient or food type:
storing a taste and/or smell modifying substance in a reservoir that is configured to be attached to or implanted within a person's body;
detecting when the person is digesting a selected nutrient or food type within their mouth;
allowing the person to digest a selected amount of the selected nutrient or food type, during a period of time, without modifying the taste and/or smell of the selected nutrient or food type; and
modifying the taste and/or smell of the selected nutrient or food type in the person's mouth if the person digests more than the selected amount of the selected nutrient or food type.
